(19) United States
(12) Patent Application Publication
(10) Pub. No.: US 2008/0260818 A1
(43) Pub. Date: Oct. 23, 2008

(54) Controlled Absorption of Statins in the Intestine

(75) Inventors: Adel Penhasi, Holon (IL); Marina Ruderman, Zichron Yaakov (IL); Maxim Gomberg, Jerusalem (IL)

Correspondence Address: WINSTON & STRAWN LLP;PATENT DEPARTMENT, 1700 K STREET, N.W., WASHINGTON, DC, 20006, US

(73) Assignee: DEXCEL PHARMA TECHNOLOGIES LTD., Hadera (IL)

(21) Appl. No.: 11/909,961

(22) PCT Filed: Mar. 27, 2006
(86) PCT No.: PCT/IL2006/000387

§ 371 Date: Apr. 23, 2008

Related U.S. Application Data
(60) Provisional application No. 60/665,362, filed on Mar. 28, 2005.

(51) Int. Cl. A61K 9/24; A61K 9/16; A61K 9/22; A61K 9/52; A61K 9/28; A61K 9/32; A61K 9/36; A61K 9/56; A61K 9/58; A61K 9/62

(52) U.S. Cl. 424/457; 424/472; 424/494; 424/497; 514/460

(57) ABSTRACT

The present invention provides a controlled absorption formulation in which modified release of active ingredient preferentially occurs in the lower gastrointestinal tract, including the colon. The formulation supports a significantly higher bioavailability of the active ingredient into the body of the subject than can be achieved from the currently used conventional formulation, such that therapeutically significant plasma levels of statin are maintained for an extended period after administration. The formulation preferably features a core over which an outer coating is layered. The core is optionally and preferentially in the form of a tablet.

### FIELD OF THE INVENTION

The present invention relates to a formulation for the controlled absorption of a medication, and in particular, to a formulation for the delayed onset, controlled release of HMG-CoA reductase inhibitors (statins) predominantly in the lower GI tract.

### BACKGROUND OF THE INVENTION

Controlled release formulations for oral administration of drugs are beneficial for a number of reasons. For example, they enable the patient to ingest the formulation less frequently, which may lead to increased patient compliance with the dosing regimen. They may also result in fewer side effects, as peaks and troughs of the level of the drug in the bloodstream of the patient may be decreased, leading to a more even drug level in the blood over a period of time. Such formulations may also provide a longer plateau concentration of the drug in the blood. The size and frequency of dosing is determined by the pharmacodynamic and pharmacokinetic properties of the drug. The slower the rate of absorption, the less the blood concentrations fluctuate within a dosing interval. This enables higher doses to be given less frequently. For drugs with relatively short half-lives, the use of modified-release products may maintain therapeutic concentrations over prolonged periods.

Currently, delayed onset, controlled release drug delivery systems administered by the oral route are usually based on either a gel forming matrix or coated formulations, or the combination thereof.

A delayed onset drug delivery system should preferentially deliver drugs to any part of the lower GI tract, as a site for topical delivery and subsequent absorption of the drug. This concept relies on the fact that the retention time of the drug delivery system through the colon may be the longest as compared to other parts of gastrointestinal tract. Likewise, such a delivery system could also advantageously use the unique continuous absorption characterizing the colon, which results in flatter, more consistent concentration levels of the drug in blood. Such absorption, of course, can contribute significantly to reduction of the fluctuations in blood drug concentration thus preventing the side effects which may appear upon using either immediate or conventional controlled release formulations, thereby improving compliance.

Many different types of delayed onset formulations for delivery to the colon are known in the art. These include pH-dependent delivery systems; pH-independent delivery systems, including systems depending on factors such as hydrolytic degradation, hydrolysis, enzymatic degradation, and physical degradation, such as dissolution; and time-dependent delivery systems. Time-dependent systems release their drug load after a preprogrammed time delay. To attain colonic release, the lag time should equal the time taken for the system to reach the colon. The small intestinal transit time is generally considered to be in the region of three to four hours.

The statins are a class of compounds which contain a moiety that can exist as either a 3-hydroxy lactone ring or as the corresponding open ring dihydroxy acid. The structural formulas of these and additional HMG-CoA reductase inhibitors, are described in M. Yalpani, “Cholesterol Lowering Drugs”, Chemistry & Industry, pp. 85-89 (1996).

The statins are orally effective in the reduction of serum cholesterol levels, by competitively inhibiting 3-hydroxy-3-methylglutaryl coenzyme A (HMG CoA) reductase, and play an important role in primary and secondary prevention of ischemic heart disease and myocardial infarct.

The statins include natural fermentation products lovastatin (described in U.S. Pat. No. 4,231,938) and mevastatin (described in U.S. Pat. No. 3,671,523); as well as a variety of semi-synthetic and totally synthetic products, which include simvastatin (U.S. Pat. No. 4,444,784); pravastatin sodium salt (U.S. Pat. No. 4,346,227); fluvastatin sodium salt (U.S. Pat. No. 5,354,772); atorvastatin calcium salt (U.S. Pat. No. 5,273,995); and cerivastatin sodium salt (also known as rivastatin; U.S. Pat. No. 5,177,080).

An osmosis-controlled release formulation for a statin is taught in U.S. Pat. No. 5,916,595, to Andrx which comprises a core containing a water swellable polymer and an osmotic agent, a channeling agent and a water insoluble cellulose polymer. Water is drawn into the tablet, which expands to the point where the outer coating fails in one particular area to form a constricted opening which releases the internal contents of the tablet which contain the drug. Thereafter, the aqueous medium of the tablet shell continues to release the drug as it dissolves until the osmotic pressure inside the tablet shell equals that of the surrounding environment. At the late stages of the in vivo release, the tablet shell collapses and/or disintegrates completely in order to substantially release the remaining drug. Complete release occurs over a period of 4-30 h.

U.S. Pat. No. 5,882,682 to Merck teaches controlled delivery of simvastin from a core by use of a water insoluble coating which contains apertures. The release rate of the simvastatin is a function of the number and size of the apertures in the coating, and again is a slow, extended form of release.

U.S. Pat. No. 4,997,658 to Merck teaches a method for lowering plasma cholesterol by using a HMG-CoA reductase inhibitor in a sustained release manner over a period of 6-24 hours as a slow, extended form of release, thereby reducing the amount of HMG-CoA reductase inhibitor circulating in the bloodstream.

WO 01/34123 to Andrx teaches a controlled release dosage form for a drug which may include the statins, in which the release is gradual, and occurs at about 10 to about 32 hours after oral administration; again the drug emerges from the formulation in a slow, extended form of release. This dosage form is intended to provide a moderate level of plasma statin concentration, wherein the mean time to maximum plasma concentration of the drug is about 10 to 32 hours after oral administration. This application does not relate to the way by which a higher blood plasma concentration of the active material may be obtained after administration.

WO 04/021972 to Biovail discloses formulations which putatively decrease the concentration of lovastatin and simvastatin and their active metabolites in the systemic circulation and at the same time provide increased concentrations of these statins in the liver. The disclosure teaches extended release formulations as preferred over a burst release formulation, and the structure of the formulations taught may for example feature a number of compartments.

US 20030176502 to Athapharma describes controlled-release formulations of pravastatin in the small intestine, thereby limiting systemic exposure of the body to pravastatin.

WO 01/32162 describes a method comprising administration of an HMG CoA reductase inhibitor in a slow-release formulation to the small intestine that provides a clinically effective level in the portal vein and liver, but less than that required to provide a clinically effective blood level in the peripheral circulation.

WO 00/33821 to BMS describes an enteric coated pravastatin bead formulation

WO 98/15290 to Astra describes a sustained release formulation of fluvastatin.

EP1036563 describes a delayed-release oral formulation of dihydroxy open acid statin.

A gastrointestinal controlled delivery system is disclosed in U.S. Pat. Nos. 5,840,332 and 6,703,044, neither of which relate to the use of those formulations for very poorly water soluble drugs in general and make no reference whatsoever to the statins in particular.

Various references teach the metabolism and pharmacokinetics of statins in the human body (see for example M. J. Garcia et al., Clinical Pharmacokinetics of Statins, Clin. Pharmacol. 2003, 25 (6): 457-481).

Simvastatin is administered as the inactive lactone prodrug that must be hydrolyzed in the plasma and liver to the beta-hydroxy acid form for pharmacological activity. Simvastatin is believed to be metabolized in the liver and intestine, at least by the enzyme CYP3A, considering the beta-hydroxy acid form as the drug, the major active metabolites are 6-beta-hydroxymethyl and 6-beta-hydroxy simvastatin, which retain approximately 40% and 50%, respectively, of HMG-CoA reductase activity. Absorption reaches 60% while the bioavailability of the beta-hydroxy acid form following oral administration of simvastatin is less than 5%.

The poor bioavailability of simvastatin is mainly attributed to its low solubility in gastrointestinal fluids, low permeability through the mucosal membrane, and extensive first-pass metabolism. Since simvastatin (as stated above) is believed to be a CYP3A4 substrate, simvastatin may be expected to undergo significant intestinal metabolism.

The above cited reference also teaches that about 87% of the absorbed dose of simvastatin undergoes hepatic metabolism. The activation of simvastatin is by carboxyesterase-mediated hydrolysis, which occurs to a slight extent in plasma and in a higher extent in the liver. Both the parent lactone and the acid forms are normally present in very small amounts in the plasma, due to a high hepatic extraction ratio.

Simvastatin and its active acid forms are highly bound to plasma proteins, primarily to albumin (more than 95%). More than 98% of simvastatin is protein bound versus 94.5% for the open hydroxyl acid form. As only unbound drug is assumed to be able to enter the tissues, the high protein binding and low plasma concentrations of simvastatin are in agreement with the low peripheral tissue exposure in humans.

Physicians' Desk Reference 58th edition, 2004, pages 2113-2118 teaches the metabolism, pharmacokinetics, pharmacodynamics and side effects of simvastatin, and is hereby incorporated by reference as if fully set forth herein.

### SUMMARY OF THE INVENTION

The background art does not teach or suggest a delayed onset, modified release formulation for delivery of statins to the GI tract including the lower GI tract and the colon, providing an increased bioavailability as measured by AUC of a statin and/or active forms of said statin, relative to that resulting from the administration of an equivalent dose of conventional immediate release formulations.

Nor does the background teach or suggest a delayed onset, controlled release formulation, which provides greater bioavailability. The background art also does not teach or suggest such a formulation, which provides fewer side effects, for greater patient compliance and comfort.

There remains an unmet need for formulations of statins with improved bioavailability and pharmacokinetics of the active species while minimizing side effects and reduced dosage.

The present invention overcomes the deficiencies of known formulations of statins by providing a delayed onset controlled release formulation for once a day administration in which controlled release of the active ingredient preferably occurs in the lower GI tract including the colon. Alternatively, such release may occur in the small intestine. The formulation provides significant plasma levels of a statin or its metabolites that are maintained for an extended period after administration.

Without wishing to be limited by a single hypothesis, the formulation of the present invention is believed to have preferential release of the drug in the lower GI tract, resulting in increased amount of a statin and its active hydroxyl acid forms than would have been formed if the drug were allowed to be absorbed into the bloodstream prior to reaching the appropriate section(s) of the intestine.

Local intestinal production of a greater amount of the active metabolite, probably through the activity of colonic natural flora, or via other metabolic routes, will further enhance the desired clinical effect and allow the achievement of intestinal drug levels of these metabolites that are unattainable by systemic or conventional oral delivery.

By using the delayed onset controlled release formulation according to the present invention, it may be possible to obtain increased production of active forms in the gut than that which can be obtained through carboxyesterse-mediated hydrolysis in the liver.

Further advantages of at least partial colonic delivery are that statins probably have greater solubility in the colon, and colon transit times are longer, resulting in increased time of exposure of the drug, and hence greater absorption.

Orally administered drugs or chemical agents that are processed to active forms in the intestinal environment can be administered to a patient who suffers from impaired liver function. Impaired liver function prevents or diminishes the normal hepatic metabolism of drugs to active metabolites. The increased production of active forms following administration of the formulations of the present invention is believed to reduce stress on the liver. The liver enzyme CYP3A4 is also present in the intestine, hence metabolism in the intestine can serve an alternative for metabolism in the liver for such drugs in these patients.

Another reason for delivering statin in the lower GI tract using the formulations of the present invention is to avoid high concentrations of CYP3A4, in which is largely present at a high concentration in the upper GI tract, and thereby to enable the release of statin to take place in the lower GI tract where the concentration of CYP3A4 is relatively poor. This process can increase the bioavailability of the statin.

A further reason for delivering statin in the lower GI tract using the formulations of the present invention is to reduce the potential for interaction between drugs. This is in the light of the fact that many drugs may either induce or inhibit the activity of CYP3A4, and thus the bioavailability of statin may be affected.

One of the many advantages of the formulation of the present invention is that a reduced dosage of a statin may be used in comparison to the presently available formulations, which may lead to the following beneficial effects:

1. Reduced liver side effects, such as a reduced level of transaminase for example (dose-related side effect).

2. Reduced incidence of rhabdomyolysis, muscle pain, and/or reduced level of CPK (dose-related side effect).

3. Reduced gastrointestinal effects including but not limited to nausea, dyspepsia, flatulence, and/or constipation (may be dose related side effects).

4. Better tolerated multiple drug treatment in which at least one additional drug is metabolized by the liver.

A further advantage of the present invention is that a reduced food effect on the release may be obtained, since the formulation according to the present invention provides a release occurring predominantly in the lower gastrointestinal tract including the colon. Metabolism and absorption of orally administered drugs are commonly known to be affected by interactions with food. The formulation of the present invention is expected to be little affected or even unaffected by such interactions, since metabolism and absorption of the statin occurs in the intestine, optionally and preferably in the colon.

According to a first aspect, the formulation according to the present invention provides a delayed onset controlled release formulation for drug release of a statin in the gastrointestinal tract comprising a core, over which an outer coating is layered.

According to one embodiment, the core is preferably in the form of a tablet.

According to other embodiments, the core may be selected from the group consisting of pellets, microparticles, agglomerates, capsule or any other solid dosage form.

According to one embodiment the present invention provides a delayed onset controlled release formulation for drug release of a statin in the gastrointestinal tract comprising a core comprising at least one statin, wherein the core includes at least one release controlling agent and an outer coating over the core the outer coating comprising a polymer that erodes and/or is ruptured after a predetermined period of time post administration.

According to various alternative embodiments, the core is selected from the group consisting of a compressed tablet, pellets, microparticles, agglomerates, and capsules. According to various embodiments the statin is selected from lovastatin, mevastatin simvastatin, pravastatin, fluvastatin, atorvastatin, and cerivastatin also known as rivastatin, and salts thereof. The dosage levels of the active ingredient may easily be determined by one of ordinary skill in the art. According to certain currently preferred embodiments the statin is selected from simvastatin atorvastatin and lovastatin.

According to preferred embodiments of the present invention, there is provided a delayed onset slow release formulation for a statin and/or active forms of said statin, comprising: a slowly disintegrating core comprising at least one statin, a filler and a release controlling agent, wherein said release controlling agent is present in a range of from about 5% to about 20% weight per weight relative to the total weight of the core, and a rate-controlling outer coating over said core, said rate-controlling outer coating featuring a mixture of cellulosic water insoluble polymers.

Optionally, the mixture of cellulosic water insoluble polymers comprises any two or more polymers selected from the group consisting of HPMC, EC, and microcrystalline cellulose.

According to preferred embodiments of the present invention, there is provided a delayed onset slow release formulation for a statin and/or active forms of said statin, comprising: a slowly disintegrating core comprising at least one statin, a filler and a release controlling agent, wherein said release controlling agent is present in a range of from about 5% to about 20% weight by weight of the total weight of said core, and a rate-controlling outer coating over said core, said rate-controlling outer coating featuring a combination of a water insoluble polymer and a water soluble polymer.

Optionally said combination of said water insoluble polymer and said water soluble polymer comprises a water insoluble polymer selected from the group consisting of a podimethylaminoethylacrylate/ethylmethacrylate copolymer, the copolymer being based on acrylic and methacrylic acid esters with a low content of quaternary ammonium groups, wherein the molar ratio of the ammonium groups to the remaining neutral (meth)acrylic acid esters is approximately 1:20, the polymer corresponding to USP/NF “Ammonio Methacrylate Copolymer Type A”, an ethylmethacrylate/chlorotrimethylammoniumethyl methacrylate copolymer, the copolymer based on acrylic and methacrylic acid esters with a low content of quaternary ammonium groups wherein the molar ratio of the ammonium groups to the remaining neutral (meth)acrylic acid esters is 1:40, the polymer corresponding to USP/NF “Ammonio Methacrylate Copolymer Type B”, a dimethylaminoethylmethacrylate/methylmethacrylate and butylmethacrylate copolymer, a copolymer based on neutral methacrylic acid esters and dimethylaminoethyl methacrylate esters wherein the polymer is cationic in the presence of acids, an ethylacrylate and methylacrylate/ethylmethacrylate and methyl methylacrylate copolymer, the copolymer being a neutral copolymer based on neutral methacrylic acid and acrylic acid esters, ethylcellulose, shellac, zein, and waxes, paraffin, cellulose acetate, cellulose propionate, cellulose acetate propionate, cellulose acetate butyrate, cellulose acetate phthalate, cellulose triacetate, poly (methyl methacrylate), poly(ethylmethacrylate), poly (butyl methacrylate), poly (isobutyl methacrylate), and poly (hexyl methacrylate), poly (isodecyl methacrylate), poly(lauryl methacrylate), poly (phenyl methacrylate), poly (methylacrylate), poly (isopropyl acrylate), poly (isobutyl acrylate) poly(octadecyl acrylate), poly (ethylene), poly (ethylene) low density, poly(ethylene) high density, poly (ethylene oxide), poly (ethyleneterephthalate), poly (vinyl isobutyl ether), poly (vinyl acetate), poly(vinyl chloride) and polyurethane, and/or mixtures thereof.

Preferably, said water insoluble polymer comprises ethylcellulose.

Also preferably, said water soluble polymer comprises polyvinyl alcohol, polyvinylpyrrolidone (PVP), copolyvidone, methylcellulose, hydroxypropylcellulose, hydroxypropylmethyl cellulose, polyethylene glycol, carboxymethyl cellulose (sodium salt), hydroxyethyl cellulose, a water soluble gum, polysaccharide and/or mixtures thereof. More preferably said water soluble polymer comprises copolyvidone.

According to preferred embodiments of the present invention, there is provided a delayed onset slow release formulation for a statin and/or active forms of said statin, comprising: a slowly disintegrating core comprising at least one statin, a filler and a release controlling agent, wherein said release controlling agent is present in a range of from about 5% to about 20% weight by weight of the total weight of said core, and a rate-controlling outer coating over said core, said rate-controlling outer coating featuring an enteric polymer coating.

Preferably, said enteric polymer coating comprises a polymer selected from the group consisting of cellulose acetate phthalate, hydroxy propyl methyl cellulose acetate succinate, poly(methacrylic acid, methyl methacrylate)1:1 and (Eudragit L100), poly(methacrylic acid, ethyl acrylate)1:1 (Eudragit L30D-55).

More preferably, said polymer comprises HPMC AS.

According to preferred embodiments of the present invention, there is provided a delayed onset slow release formulation for a statin and/or active forms of said statin, comprising: a slowly disintegrating core comprising at least one statin, a filler and a release controlling agent, wherein said release controlling agent is present in a range of from about 5% to about 20% weight by weight of the total weight of said core, and a rate-controlling outer coating over said core, said rate-controlling outer coating featuring a polymer disintegrating at pH values above about pH 5.0.

Preferably said enteric polymer coating comprises a polymer selected from the group consisting of cellulose acetate phthalate, hydroxy propyl methyl cellulose acetate succinate, poly(methacrylic acid, methyl methacrylate)1:1 and (Eudragit L100), poly(methacrylic acid, ethyl acrylate)1:1 (Eudragit L30D-55).

More preferably said polymer comprises HPMC AS.

According to preferred embodiments of the present invention, there is provided a delayed onset slow release formulation for a statin and/or active forms of said statin, comprising: a slowly disintegrating core comprising at least one statin, a filler and a release controlling agent, wherein said release controlling agent is present in a range of from about 5% to about 20% weight by weight of the total weight of said core, and a rate-controlling outer coating over said core, said rate-controlling outer coating featuring a cellulosic polymer that is applied as a dry coating, said polymer forming a hydrogel.

Preferably, said cellulosic polymer of said dry coating comprises HPMC K 15 M.

According to preferred embodiments of the present invention, there is provided a delayed onset slow release formulation for a statin and/or active forms of said statin, comprising: a slowly disintegrating core comprising at least one statin, a filler and a release controlling agent, wherein said release controlling agent is present in a range of from about 5% to about 20% weight by weight of the total weight of said core, and a rate-controlling outer coating over said core, said rate-controlling outer coating featuring water insoluble hydrophilic particles embedded in a water insoluble flexible polymer, wherein said statin and/or active forms of said statin are released through diffusion through said water insoluble hydrophilic particles.

Preferably said water insoluble flexible polymer comprises a methacrylic polymer. More preferably, said methacrylic polymer comprises Eudragit E. Most preferably, said water insoluble hydrophilic particles comprise a polymer forming a hydrogel upon contact with liquid. Preferably, said hydrogel forming polymer is selected from the group consisting of poly(hydroxy alkyl methacrylate); kappa-carrageenan; polyvinylpyrrolidone; anionic and cationic hydrogels; polyelectrolyte complexes; poly(vinyl alcohol) having low amounts of acetate, cross-linked with glyoxal, formaldehyde, or glutaraldehyde; a mixture comprising methyl cellulose, cross-linked agar and carboxymethyl cellulose; a water-insoluble, water-swellable copolymer produced by forming a dispersion of finely divided maleic anhydride with styrene, ethylene, propylene, butylene or isobutylene; water-swellable polymers of N-vinyl lactams; polysaccharide, water swellable gums, high viscosity hydroxylpropylmethyl cellulose and/or mixtures thereof.

Preferably, said water insoluble polymer is selected from the group consisting of cross-linked polysaccharide, water insoluble starch, microcrystalline cellulose, water insoluble cross-linked peptide, water insoluble cross-linked protein, water insoluble cross-linked gelatin, water insoluble cross-linked hydrolyzed gelatin, water insoluble cross-linked collagen, modified cellulose, and cross-linked polyacrylic acid.

More preferably, said cross-linked polysaccharide is selected from the group consisting of insoluble metal salts or cross-linked derivatives of alginate, pectin, xantham gum, guar gum, tragacanth gum, and locust bean gum, carrageenan, metal salts thereof, and covalently cross-linked derivatives thereof.

Most preferably, said modified cellulose is selected from the group consisting of cross-linked derivatives of hydroxypropylcellulose, hydroxypropylmethylcellulose, hydroxyethylcellulose, methylcellulose, carboxymethylcellulose, and metal salts of carboxymethylcellulose. Most preferably, said water insoluble polymer is calcium pectinate.

Alternatively, said water insoluble polymer is microcrystalline cellulose.

According to preferred embodiments of the present invention, there is provided a delayed onset slow release formulation for a statin and/or active forms of said statin, comprising: a slowly disintegrating core comprising at least one statin, a filler and a release controlling agent, wherein said release controlling agent is present in a range of from about 5% to about 20% weight by weight of the total weight of said core, and a rate-controlling outer coating over said core, said rate-controlling outer coating featuring a dry coating or an enteric coating, with the proviso that said core does not comprise a disintegrating agent.

Preferably said filler is present in an amount ranging from about 10% to about 85% (W/W) relative to the total weight of core.

Optionally said filler is selected from the group consisting of starch, lactitol, lactose, an inorganic calcium salt, microcrystalline cellulose, sucrose, and combinations thereof. Preferably, said filler comprises microcrystalline cellulose. More preferably, said microcrystalline cellulose has a particle size of less than about 100 microns. Most preferably said microcrystalline cellulose has a particle size of about 50 microns.

Optionally said release controlling agent comprises a water insoluble, hydrophilic swellable polymer. Preferably, said polymer is selected from the group consisting of poly(hydrox alkyl methacrylate); kappa-carrageenan; polyvinylpyrrolidone; anionic and cationic hydrogels; polyelectrolyte complexes; poly(vinyl alcohol) having low amounts of acetate, cross-linked with glyoxal, formaldehyde, or glutaraldehyde; a mixture comprising methyl cellulose, cross-linked agar and carboxymethyl cellulose; a water-insoluble, water-swellable copolymer produced by forming a dispersion of finely divided maleic anhydride with styrene, ethylene, propylene, butylene or isobutylene; water-swellable polymers of N-vinyl lactams; polysaccharide, water swellable gums, high viscosity hydroxylpropylmethyl cellulose and/or mixtures thereof. More preferably, said water insoluble polymer is selected from the group consisting of cross-linked polysaccharide, water insoluble starch, microcrystalline cellulose, water insoluble cross-linked peptide, water insoluble cross-linked protein, water insoluble cross-linked gelatin, water insoluble cross-linked hydrolyzed gelatin, water insoluble cross-linked collagen, modified cellulose, and cross-linked polyacrylic acid. Optionally, said cross-linked polysaccharide is selected from the group consisting of insoluble metal salts or cross-linked derivatives of alginate, pectin, xantham gum, guar gum, tragacanth gum, and locust bean gum, carrageenan, metal salts thereof, and covalently cross-linked derivatives thereof.

Optionally, said modified cellulose is selected from the group consisting of cross-linked derivatives of hydroxypropylcellulose, hydroxypropylmethylcellulose, hydroxyethylcellulose, methylcellulose, carboxymethylcellulose, and metal salts of carboxymethylcellulose.

Preferably, said water insoluble polymer is calcium pectinate.

Alternatively, said water insoluble polymer is microcrystalline cellulose.

According to preferred embodiments of the present invention, there is provided a delayed onset controlled release formulation for providing an increased bioavailability as measured by AUC of a statin and/or active forms of said statin, relative to that resulting from the administration of an equivalent dose of the conventional immediate release formulations, comprising: a slowly disintegrating core comprising at least one statin and at least one release controlling agent and a rate-controlling outer coating over said core, providing controlled release.

Preferably, said controlled release is selected from the group consisting of delayed sustained release, delayed controlled release, delayed slow release, delayed prolonged release and delayed extended release.

Optionally said release controlling agent comprises a material selected from the group consisting of a suitable hydrophilic cellulosic gelling polymer; vinyl polymers; acrylic polymers and copolymers, natural and synthetic gums, gelatin, collagen, proteins, polysaccharides; and mixtures thereof. Preferably, said polysaccharide in said release controlling agent comprises a cross-linked polysaccharide. More preferably, said cross-linked polysaccharide is selected from the group consisting of insoluble metal salts or cross-linked derivatives of alginate, pectin, xantham gum, guar gum, tragacanth gum, and locust bean gum, carrageenan, metal salts thereof, and covalently cross-linked derivatives thereof.

Optionally said cellulosic polymer comprises a modified cellulose. Preferably, said modified cellulose is selected from the group consisting of cross-linked derivatives of hydroxypropylcellulose, hydroxypropylmethylcellulose, hydroxyethylcellulose, methylcellulose, carboxymethylcellulose, and metal salts of carboxymethylcellulose.

Optionally, said cellulosic gelling polymer comprises a material selected from the group consisting of methylcellulose, carboxymethylcellulose, hydroxypropylcellulose, hydroxypropylmethylcellulose, hydroxyethylcellulose. Preferably, said swellable polymeric coating layer comprises hydroxypropylmethylcellulose.

Optionally said vinyl polymer is selected from the group consisting of polyvinylpyrrolidone, polyvinyl alcohol, and the like.

Also optionally said acrylic polymers and copolymers are selected from the group consisting of acrylic acid polymer, methacrylic acid copolymers, ethyl acrylate-methyl methacrylate copolymers.

Also optionally said natural and synthetic gums are selected from the group consisting of guar gum, arabic gum, xanthan gum.

Also optionally said polysaccharides are selected from the group consisting of pectin, pectic acid, alginic acid, sodium alginate, polyaminoacids, polyalcohols, polyglycols; and the like.

Preferably said formulation preferentially releases statin in the intestine of the subject.

More preferably, said formulation preferentially release statin in the lower gastrointestinal tract.

Most preferably, said formulation preferentially releases statin in the colon of the subject.

Optionally, said release controlling agent is present in an amount ranging from about 5% to about 20% weight per weight of said core.

Preferably, the formulation further comprises a filler. Optionally, said filler is selected from the group consisting of, starch, lactitol, lactose, an inorganic calcium salt, microcrystalline cellulose, sucrose, and combinations thereof. Preferably, said filler comprises microcrystalline cellulose. More preferably, said microcrystalline cellulose has a particle size of less than about 100 microns. Most preferably, said microcrystalline cellulose has a particle size of about 50 microns. Also most preferably, said filler is present in an amount in a range of from about 10% to about 85% (W/W) relative to the total weight of core.

Optionally, said core further comprises at least one of an absorption enhancer, a binder, a disintegrant and a hardness enhancing agent. Preferably said disintegrant is selected from the group consisting of cross-linked polyvinylpyrrolidinone, sodium starch glycolate, cross-linked sodium carboxymethylcellulose, pregelatinized starch, microcrystalline starch, water insoluble starch, calcium carboxymethylcellulose, magnesium aluminium silicate, and combinations thereof.

Preferably, said binder is selected from the group consisting of starch, polyvinylpyrrolidone, low molecular weight hydroxypropylcellulose, low molecular weight hydroxypropylmethylcellulose, low molecular weight carboxymethylcellulose, ethylcellulose, gelatin, polyethylene oxide, acacia, dextrin, magnesium aluminum silicate, and polymethacrylates. More preferably, said disintegrant is croscarmellose sodium.

Preferably said hardness enhancing agent is microcrystalline cellulose.

Optionally said core further comprises a wicking agent. Preferably said wicking agent is selected from the group consisting of colloidal silicon dioxide, kaolin, titanium oxide, fumed silicon dioxide, alumina, niacinamide, sodium lauryl sulfate, low molecular weight polyvinyl pyrrolidone, m-pyrol, bentonite, magnesium aluminum silicate, polyester, polyethylene, or mixtures thereof.

Optionally said core further comprises a stabilizer. Preferably said stabilizer is selected from the group consisting of butyl hydroxyanisole, ascorbic acid and citric acid or mixtures thereof.

Optionally said core further comprises a flow regulating agent. Preferably said flow regulating agent includes at least one of colloidal silicon dioxide and aluminum silicate.

Optionally said core further comprises a lubricant. Preferably said lubricant is selected from the group consisting of stearate salts; stearic acid, talc, sodium stearyl fumarate, sodium lauryl sulfate, polyethylene glycol, and glycerol behenate, or a combination thereof. More preferably said lubricant is magnesium stearate.

Optionally said core further comprises an antioxidant. Preferably said antioxidant is selected from the group consisting of 4,4 (2,3 dimethyl tetramethylene dipyrochatechol), Tocopherol-rich extract (natural vitamin E), α-tocopherol (synthetic Vitamin E), β-tocopherol, γ-tocopherol, δ-tocopherol, Butylhydroxinon, Butyl hydroxyanisole (BHA), Butyl hydroxytoluene (BHT), Propyl Gallate, Octyl gallate, Dodecyl Gallate, Tertiary butylhydroquinone (TBHQ), Fumaric acid, Malic acid, Ascorbic acid (Vitamin C), Sodium ascorbate, Calcium ascorbate, Potassium ascorbate, Ascorbyl palmitate, Ascorbyl stearate, Citric acid, Sodium lactate, Potassium lactate, Calcium lactate, Magnesium lactate, Anoxomer, Erythorbic acid, Sodium erythorbate, Erythorbin acid, Sodium erythorbin, Ethoxyquin, Glycine, Gum guaiac, Sodium citrates (monosodium citrate, disodium citrate, trisodium citrate), Potassium citrates (monopotassium citrate, tripotassium citrate), Lecithin, Polyphosphate, Tartaric acid, Sodium tartrates (monosodium tartrate, disodium tartrate), Potassium tartrates (monopotassium tartrate, dipotassium tartrate), Sodium potassium tartrate, Phosphoric acid, Sodium phosphates (monosodium phosphate, disodium phosphate, trisodium phosphate), Potassium phosphates (monopotassium phosphate, dipotassium phosphate, tripotassium phosphate), Calcium disodium ethylene diamine tetra-acetate (Calcium disodium EDTA), Lactic acid, Trihydroxy butyrophenone and Thiodipropionic acid. More preferably said antioxidant is butyl hydroxyanisole.

Optionally said core further comprises a chelating agent. Preferably said chelating agent is selected from the group consisting of antioxidants, dipotassium edentate, disodium edentate, edetate calcium disodium, edetic acid, fumaric acid, malic acid, maltol, sodium edentate, trisodium edetate.

Optionally said core further comprises a sequestrate. Preferably said sequestrate is selected from the group consisting of citric acid and ascorbic acid.

Optionally said coating comprises at least one of the following: a. pH dependent polymer; b. a combination of at least one water soluble polymer and at least one water insoluble polymer; c. a combination of at least one swellable polymer and at least one water insoluble polymer; d. a combination of at least a water soluble pore forming agent and at least one water insoluble polymer; e. at least one swellable gel forming polymer; g. an erodible composition; h. a combination of at least one pH dependent polymer and at least one water insoluble polymer.

Optionally said pH dependent polymer is selected from the group consisting of a hydroxypropylmethyl cellulose phthalate, polyvinyl acetate phthalate, cellulose acetate phthalate, hydroxypropylmethyl cellulose acetate succinate, poly(methacrylic acid, methyl methacrylate) 1:1 and poly(methacrylic acid, ethyl acrylate) 1:1, alginic acid, and sodium alginate. Optionally said water-soluble polymer is selected from the group consisting of polyvinyl alcohol, polyvinylpyrrolidone (PVP), methylcellulose, hydroxypropylcellulose, hydroxypropylmethyl cellulose, or polyethylene glycol, carboxymethyl cellulose (sodium salt), hydroxyethyl cellulose, a water soluble gum, polysaccharide and/or mixtures thereof.

Optionally said water insoluble polymer is selected from the group consisting of a podimethylaminoethylacrylate/ethylmethacrylate copolymer, an ethylmethacrylate/chlorotrimethylammoniumethyl methacrylate copolymer, a dimethylaminoethylmethacrylate/methylmethacrylate and butylmethacrylate copolymer, a copolymer based on neutral methacrylic acid esters and dimethylaminoethyl methacrylate esters, an ethylacrylate and methylacrylate/ethylmethacrylate and methyl methylacrylate copolymer, ethylcellulose, shellac, zein, and waxes, paraffin, cellulose acetate, cellulose propionate, cellulose acetate propionate, cellulose acetate butyrate, cellulose acetate phthalate, cellulose triacetate, poly (methyl methacrylate), poly(ethylmethacrylate), poly (butyl methacrylate), poly (isobutyl methacrylate), and poly (hexyl methacrylate), poly (isodecyl methacrylate), poly(lauryl methacrylate), poly (phenyl methacrylate), poly (methylacrylate), poly (isopropyl acrylate), poly (isobutyl acrylate) poly(octadecyl acrylate), poly (ethylene), poly (ethylene) low density, poly(ethylene) high density, poly (ethylene oxide), poly (ethylene terephthalate), poly (vinyl isobutyl ether), poly (vinyl acetate), poly(vinyl chloride) and polyurethane, and/or mixtures thereof. Preferably said water insoluble polymer is ethylcellulose.

Optionally, said pore-forming agent is selected from the group consisting of saccharose, sodium chloride, potassium chloride, polyvinylpyrrolidone, and/or polyethyleneglycol, water soluble organic acids, sugars and sugar alcohol.

Also optionally said pore forming compound is distributed uniformly throughout said water insoluble polymer. Optionally said pore forming compound is distributed randomly throughout said water insoluble polymer.

Preferably said erodible composition comprises at least one of a slow dissolving and a slow disintegrating composition. Optionally said erodible composition comprises at least one of a slowly water soluble polymer and a swellable polymer.

Preferably said erodible composition further comprises a disintegrant.

Optionally said swellable gel-forming polymer is selected from the group consisting of cellulosic polymers; vinyl polymers; acrylic polymers and copolymers, methacrylic acid copolymers, ethyl acrylate-methyl methacrylate copolymers, natural and synthetic gums, gelatin, collagen, proteins, polysaccharides, pectin, pectic acid, alginic acid, sodium alginate, polyaminoacids, polyalcohols, polyglycols; and mixtures thereof. Preferably said cellulosic polymer is selected from the group consisting of methylcellulose, carboxymethylcellulose, hydroxypropylcellulose, hydroxypropylmethylcellulose, hydroxymethylcellulose and hydroxyethylcellulose.

Optionally said coating further comprises at least one of a lubricant, a flow promoting agent, a plasticizer, an anti-sticking agent, surfactant, wetting agent, suspending agent and dispersing agent.

Optionally said coating is a compression coating. Preferably said coating comprises a gum selected from the group consisting of xanthan gum, locust bean gum, galactans, mannans, alginates, gum karaya, pectin, agar, tragacanth, accacia, carrageenan, tragacanth, chitosan, agar, alginic acid, hydrocolloids acacia catechu, salai guggal, indian bodellum, copaiba gum, asafetida, cambi gum, Enterolobium cyclocarpum, mastic gum, benzoin gum, sandarac, gambier gum, butea frondosa (Flame of Forest Gum), myrrh, konjak mannan, guar gum, welan gum, gellan gum, tara gum, locust bean gum, carageenan gum, glucomannan, galactan gum, sodium alginate, tragacanth, chitosan, xanthan gum, deacetylated xanthan gum, pectin, sodium polypectate, gluten, karaya gum, tamarind gum, ghatti gum, Accaroid/Yacca/Red gum, dammar gum, juniper gum, ester gum, ipil-ipil seed gum, gum talha (acacia seyal), and cultured plant cell gums including those of the plants of the genera: acacia, actinidia, aptenia, carbobrotus, chickorium, cucumis, glycine, hibiscus, hordeum, letuca, lycopersicon, malus, medicago, mesembryanthemum, oryza, panicum, phalaris, phleum, poliathus, polycarbophil, sida, solanum, trifolium, trigonella, Afzelia africana seed gum, Treculia africana gum, detarium gum, cassia gum, carob gum, Prosopis africana gum, Colocassia esulenta gum, Hakea gibbosa gum, khaya gum, scleroglucan, zea, mixtures of any of the foregoing.

Preferably said compression coating comprises at least one of a heteropolysaccharide and a homopolysaccharide, or a mixture thereof. More preferably said heteropolysaccharide is xanthan gum.

Optionally said outer coating further comprises a plasticizer. Preferably said plasticizer is selected from the group consisting of dibutyl sebacate, polyethylene glycol and polypropylene glycol, dibutyl phthalate, diethyl phthalate, triethyl citrate, tributyl citrate, acetylated monoglyceride, acetyl tributyl citrate, triacetin, dimethyl phthalate, benzyl benzoate, butyl and/or glycol esters of fatty acids, refined mineral oils, oleic acid, castor oil, corn oil, camphor, glycerol and sorbitol or a combination thereof.

Optionally said coating further comprises a stiffening agent. Preferably said stiffening agent comprises cetyl alcohol.

Optionally said coating and/or said core further comprises at least one of a wetting agent, a suspending agent, and a dispersing agent, or a combination thereof. Preferably said wetting agent is selected from the group consisting of poloxamer, polyoxyethylene ethers, polyoxyethylene sorbitan fatty acid esters, polyoxymethylene stearate, sodium lauryl sulfate, sorbitan fatty acid esters, benzalkonium chloride, polyethoxylated castor oil, and docusate sodium.

Preferably said suspending agent is selected from the group consisting of alginic acid, bentonite, carbomer, carboxymethylcellulose, carboxymethylcellulose calcium, hydroxyethylcellulose, hydroxypropylcellulose, microcrystalline cellulose, colloidal silicon dioxide, dextrin, gelatin, guar gum, xanthan gum, kaolin, magnesium aluminum silicate, maltitol, medium chain triglycerides, methylcellulose, polyoxyethylene sorbitan fatty acid esters, polyvinylpyrrolidinone, propylene glycol alginate, sodium alginate, sorbitan fatty acid esters, and tragacanth.

Preferably said dispersing agent is selected from the group consisting of poloxamer, polyoxyethylene sorbitan fatty acid esters and sorbitan fatty acid esters.

Optionally said formulation further comprises an enteric coating disposed over said coating. Preferably said enteric coating is selected from the group consisting of cellulose acetate phthalate, hydroxy propyl methyl cellulose acetate succinate, poly(methacrylic acid, methyl methacrylate)1:1 and (Eudragit L100), poly(methacrylic acid, ethyl acrylate)1:1 (Eudragit L30D-55).

According to preferred embodiments of the present invention, there is provided a delayed onset controlled release formulation for providing an increased bioavailability of a statin and/or active forms of said statin as measured by AUC, relative to that resulting from the administration of an equivalent dose of the conventional immediate release formulations, comprising: a swellable, slowly disintegrating core comprising at least one statin, at least one, hydrophilic, swellable, hydrogel-forming material and a wicking controlling agent, and a rate controlling outer coating over said core comprising a water permeable agent through which fluid enters said core.

Optionally said water permeable agent comprises at least one of a water insoluble but water permeable polymer; a pore forming agent; a combination of a water insoluble polymer and a water soluble polymer; and a combination of water insoluble but flexible polymer and water insoluble but hydrophilic particles. Preferably said water insoluble but water permeable polymer comprises material selected from the group consisting of cross-linked polysaccharide, water insoluble starch, microcrystalline cellulose, water insoluble cross-linked peptide, water insoluble cross-linked protein, water insoluble cross-linked gelatin, water insoluble cross-linked hydrolyzed gelatin, water insoluble cross-linked collagen, modified cellulose, and cross-linked polyacrylic acid. Most preferably, the cross-linked polysaccharide is selected from the group consisting of insoluble metal salts or cross-linked derivatives of alginate, pectin, xantham gum, guar gum, tragacanth gum, and locust bean gum, carrageenan, metal salts thereof, and covalently cross-linked derivatives thereof.

Optionally said combination of a water insoluble polymer and a water soluble polymer comprises a water soluble polymer selected from the group consisting of polyvinyl alcohol, polyvinylpyrrolidone (PVP), copolyvidone (a (6:4) copolymer of a chain-structured vinyl pyrrolidone and vinyl acetate), methylcellulose, hydroxypropylcellulose, hydroxypropylmethyl cellulose, polyethylene glycol, carboxymethyl cellulose (sodium salt), hydroxyethyl cellulose, a water soluble gum, polysaccharide and/or mixtures thereof. Preferably said combination of a water insoluble polymer and a water soluble polymer comprises a water insoluble polymer selected from the group consisting of a podimethylaminoethylacrylate/ethylmethacrylate copolymer, the copolymer being based on acrylic and methacrylic acid esters with a low content of quaternary ammonium groups, wherein the molar ratio of the ammonium groups to the remaining neutral (meth)acrylic acid esters is approximately 1:20, the polymer corresponding to USP/NF “Ammonio Methacrylate Copolymer Type A”, an ethylmethacrylate/chlorotrimethylammoniumethyl methacrylate copolymer, the copolymer based on acrylic and methacrylic acid esters with a low content of quaternary ammonium groups wherein the molar ratio of the ammonium groups to the remaining neutral (meth)acrylic acid esters is 1:40, the polymer corresponding to USP/NF “Ammonio Methacrylate Copolymer Type B”, a dimethylaminoethylmethacrylate/methylmethacrylate and butylmethacrylate copolymer, a copolymer based on neutral methacrylic acid esters and dimethylaminoethyl methacrylate esters wherein the polymer is cationic in the presence of acids, an ethylacrylate and methylacrylate/ethylmethacrylate and methyl methylacrylate copolymer, the copolymer being a neutral copolymer based on neutral methacrylic acid and acrylic acid esters, ethyl cellulose, shellac, zein, and waxes, paraffin, cellulose acetate, cellulose propionate, cellulose acetate propionate, cellulose acetate butyrate, cellulose acetate phthalate, cellulose triacetate, poly (methyl methacrylate), poly(ethylmethacrylate), poly (butyl methacrylate), poly (isobutyl methacrylate), and poly (hexyl methacrylate), poly (isodecyl methacrylate), poly(lauryl methacrylate), poly (phenyl methacrylate), poly (methylacrylate), poly (isopropyl acrylate), poly (isobutyl acrylate) poly(octadecyl acrylate), poly (ethylene), poly (ethylene) low density, poly(ethylene) high density, poly (ethylene oxide), poly (ethyleneterephthalate), poly (vinyl isobutyl ether), poly (vinyl acetate), poly(vinyl chloride) and polyurethane, and/or mixtures thereof.

Most preferably the water insoluble polymer is ethylcellulose and the water soluble polymer is copolyvidone.

Optionally the coating comprises a film forming polymer and said pore forming agent comprises one or more of saccharose, sodium chloride, potassium chloride, polyvinylpyrrolidone, and/or polyethyleneglycol, water soluble organic acids, sugars and sugar alcohol.

Preferably said pore-forming agent is uniformly or randomly distributed throughout said film forming polymer. More preferably said pore forming agent comprises about 1 part to about 35 parts for each about 1 to about 10 parts of the film forming polymers.

Optionally said film forming polymer comprises a water insoluble polymer selected from the group consisting of a podimethylaminoethylacrylate/ethylmethacrylate copolymer, the copolymer being based on acrylic and methacrylic acid esters with a low content of quaternary ammonium groups, wherein the molar ratio of the ammonium groups to the remaining neutral (meth)acrylic acid esters is approximately 1:20, the polymer corresponding to USP/NF “Ammonio Methacrylate Copolymer Type A”, an ethylmethacrylate/chlorotrimethylammoniumethyl methacrylate copolymer, the copolymer based on acrylic and methacrylic acid esters with a low content of quaternary ammonium groups wherein the molar ratio of the ammonium groups to the remaining neutral (meth)acrylic acid esters is 1:40, the polymer corresponding to USP/NF “Ammonio Methacrylate Copolymer Type B”, a dimethylaminoethylmethacrylate/methylmethacrylate and butylmethacrylate copolymer, a copolymer based on neutral methacrylic acid esters and dimethylaminoethyl methacrylate esters wherein the polymer is cationic in the presence of acids, an ethylacrylate and methylacrylate/ethylmethacrylate and methyl methylacrylate copolymer, the copolymer being a neutral copolymer based on neutral methacrylic acid and acrylic acid esters, ethyl cellulose, shellac, zein, and waxes, paraffin, cellulose acetate, cellulose propionate, cellulose acetate propionate, cellulose acetate butyrate, cellulose acetate phthalate, cellulose triacetate, poly (methyl methacrylate), poly(ethylmethacrylate), poly (butyl methacrylate), poly (isobutyl methacrylate), and poly (hexyl methacrylate), poly (isodecyl methacrylate), poly(lauryl methacrylate), poly (phenyl methacrylate), poly (methylacrylate), poly (isopropyl acrylate), poly (isobutyl acrylate) poly(octadecyl acrylate), poly (ethylene), poly (ethylene) low density, poly(ethylene) high density, poly (ethylene oxide), poly (ethyleneterephthalate), poly (vinyl isobutyl ether), poly (vinyl acetate), poly(vinyl chloride) and polyurethane, and/or mixtures thereof.

Preferably said outer coating features said combination of water insoluble but flexible polymer and water insoluble but hydrophilic particles. Optionally said water insoluble but flexible polymer comprises a methacrylic polymer. Preferably said methacrylic polymer comprises Eudragit E.

Optionally said water insoluble but hydrophilic particles comprise a non-gel-forming hydrophilic polymer upon contact with liquid. Preferably said polymer is selected from the group consisting of poly(hydrox alkyl methacrylate); kappa-carrageenan; polyvinylpyrrolidone; anionic and cationic hydrogels; polyelectrolyte complexes; poly(vinyl alcohol) having low amounts of acetate, cross-linked with glyoxal, formaldehyde, or glutaraldehyde; a mixture comprising methyl cellulose, cross-linked agar and carboxymethyl cellulose; a water-insoluble, water-swellable copolymer produced by forming a dispersion of finely divided maleic anhydride with styrene, ethylene, propylene, butylene or isobutylene; water-swellable polymers of N-vinyl lactams; polysaccharide, water swellable gums, high viscosity hydroxylpropylmethyl cellulose and/or mixtures thereof.

Optionally said water insoluble polymer is selected from the group consisting of cross-linked polysaccharide, water insoluble starch, microcrystalline cellulose, water insoluble cross-linked peptide, water insoluble cross-linked protein, water insoluble cross-linked gelatin, water insoluble cross-linked hydrolyzed gelatin, water insoluble cross-linked collagen, modified cellulose, and cross-linked polyacrylic acid. Preferably said cross-linked polysaccharide is selected from the group consisting of insoluble metal salts or cross-linked derivatives of alginate, pectin, xantham gum, guar gum, tragacanth gum, and locust bean gum, carrageenan, metal salts thereof, and covalently cross-linked derivatives thereof. Also preferably said modified cellulose is selected from the group consisting of cross-linked derivatives of hydroxypropylcellulose, hydroxypropylmethylcellulose, hydroxyethylcellulose, methylcellulose, carboxymethylcellulose, and metal salts of carboxymethylcellulose.

More preferably said water insoluble polymer is calcium pectinate.

Alternatively said water insoluble polymer is microcrystalline cellulose.

According to preferred embodiments of the present invention, there is provided a delayed onset controlled release formulation for providing an increased bioavailability of a statin and/or active forms of said statin as measured by AUC, relative to that resulting from the administration of an equivalent dose of the conventional immediate release formulations, comprising: a swellable, slowly disintegrating core comprising at least one statin and at least one release controlling agent and an outer coating over said core, providing delayed release, characterized in that the in vivo blood plasma concentration of said statin and/or a pharmaceutically acceptable salt and/or ester thereof in the subject is substantially zero for at least about two hours after oral administration.

According to preferred embodiments of the present invention, there is provided a delayed onset controlled release formulation for providing an increased bioavailability of a statin and/or active forms of said statin as measured by AUC, relative to that resulting from the administration of an equivalent dose of the conventional immediate release formulations, comprising: a core comprising at least one statin and at least one release controlling agent and an erodible film outer coating over said core, providing delayed release.

According to preferred embodiments of the present invention, there is provided a delayed onset controlled release formulation for providing an increased bioavailability of a statin and/or active forms of said statin as measured by AUC, relative to that resulting from the administration of an equivalent dose of the conventional immediate release formulations, comprising a slowly disintegrating core comprising at least one statin and at least one release controlling agent, and a pH dependent film outer coating over said core, providing delayed release.

According to preferred embodiments of the present invention, there is provided a delayed onset controlled release formulation for providing an increased bioavailability of a statin and/or active forms of said statin as measured by AUC, relative to that resulting from the administration of an equivalent dose of the conventional immediate release formulations, comprising: a slowly disintegrating core comprising at least one statin and at least one release controlling agent and an outer coating over said core, comprising a combination of a water soluble polymer and/or a water swellable hydrophilic polymer and a water insoluble polymer.

Optionally said swellable hydrophilic polymer is a gel-forming polymer. Preferably said swellable hydrophilic polymer is selected from the group consisting of poly(hydrox alkyl methacrylate); kappa-carrageenan; polyvinylpyrrolidone; anionic and cationic hydrogels; polyelectrolyte complexes; poly(vinyl alcohol) having low amounts of acetate, cross-linked with glyoxal, formaldehyde, or glutaraldehyde; a mixture including methyl cellulose, cross-linked agar and carboxymethyl cellulose; a water-insoluble, water-swellable copolymer produced by forming a dispersion of finely divided maleic anhydride with styrene, ethylene, propylene, butylene or isobutylene; water-swellable polymers of N-vinyl lactams; polysaccharide, water swellable gums, high viscosity of hydroxylpropylmethyl cellulose and/or mixtures of any of the foregoing.

Preferably the coating further comprises a plasticizer. More preferably said plasticizer is selected from the group consisting of cetylated monoglycerides, butyl phthalyl butyl glycolate, dibutyl tartrate, diethyl phthalate, dimethyl phthalate, ethyl phthalyl ethyl glycolate, glycerin, ethylene glycol, propylene glycol, triacetin citrate, triacetin, tripropinoin, diacetin, dibutyl phthalate, acetyl monoglyceride, polyethylene glycols, castor oil, triethyl citrate, polyhydric alcohols, acetate esters, glycerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-1-octyl phthalate, di-1-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, glyceryl monocaprylate, and glyceryl monocaprate.

Preferably said water soluble polymer dissolves upon exposure to fluids, thereby forming channels in said coating.

According to preferred embodiments of the present invention, the formulation releases substantially no statin in vitro for at least about 2 to about 6 hours.

Preferably said statin is released in the small intestine and/or lower gastrointestinal tract resulting in an increased intestinal active forms formation.

More preferably said statin is released in the small intestine and/or lower gastrointestinal tract resulting in an increased concentration of at least one active forms in the blood.

More preferably said formulation comprises a decreased dosage of said statin and/or said pharmaceutically acceptable salt and/or ester thereof.

Preferably said statin is selected from the group comprising simvastatin, beta-hydroxy acid simvastatin, lovastatin, mevastatin, pravastatin, fluvastatin, atorvastatin, pitavastatin, rivastatin and cerivastatin, or pharmaceutically acceptable salts and/or esters thereof. More preferably said statin comprises simvastatin.

According to preferred embodiments of the present invention, there is provided a method for using a delayed onset controlled release formulation to reduce stress on the liver of the subject treated by at least one other drug involved into the liver metabolism.

According to preferred embodiments of the present invention, there is provided a method for using a delayed onset controlled release formulation to reduce stress on the liver of the subject.

According to preferred embodiments of the present invention, there is provided a method for using a delayed onset controlled release to reduce liver side effects including increased level of transaminases.

According to preferred embodiments of the present invention, there is provided a method for using a delayed onset controlled release formulation to reduce muscle pain and level of CPK.

According to preferred embodiments of the present invention, there is provided a method for using a delayed onset controlled release formulation to reduce gastrointestinal effects comprising nausea, dyspepsia, flatulence and constipation.

According to preferred embodiments of the present invention, there is provided a method for using a delayed onset controlled release formulation, for providing release of said statin or said pharmaceutically acceptable salt or ester or active form thereof that is not affected by food.

According to preferred embodiments of the present invention, there is provided a method for using a delayed onset controlled release formulation, for providing treatment for high blood cholesterol to a subject in need thereof.

According to preferred embodiments of the present invention, there is provided a method for providing a therapeutically effective amount of a statin and/or a pharmaceutically acceptable salt and/or ester and/or active form thereof to a subject, comprising administering orally to the subject a delayed onset controlled release formulation.

According to preferred embodiments of the present invention, there is provided a method for providing a delayed burst release of a therapeutically effective amount of a statin and/or a pharmaceutically acceptable salt and/or ester thereof to a subject wherein substantially no statin is released in vitro for at least about two hours.

According to preferred embodiments of the present invention, there is provided a method for providing enhanced bioavailability of statin and/or a pharmaceutically acceptable salts and/or esters thereof and/or its related metabolite in a subject, comprising: administering orally to the subject a delayed onset controlled release formulation comprising a core and an outer coating that surrounds the core; said core comprising a statin, or a pharmaceutically acceptable salt thereof and at least one release controlling agent, and said coating characterized by at least one of a. pH dependent coating film, preferably an enteric coating; b. a combination of at least one water soluble polymer and at least one water insoluble polymer; c. a combination of at least one swellable polymer and at least one water insoluble polymer; d. a combination of at least a water soluble pore forming agent and at least one water insoluble polymer; e. at least one swellable gel forming polymer; f. at least one erodible polymer; g. a combination of at least one pH dependent polymer and at least one water insoluble polymer; h. a two-layer coating comprising a rupturable outer layer and swellable inner layer; characterized in that the in vivo blood plasma concentration of said statin is substantially zero for at least about two hours after oral administration.

According to preferred embodiments of the present invention, there is provided a method for providing an increased amount of statin and/or a pharmaceutically acceptable salt and/or ester thereof, relative to that resulting from the administration of an equivalent dose of the conventional immediate release formulations, to a subject in need thereof, comprising; administering orally to the subject a delayed onset controlled release formulation comprising a core and an outer coating that surrounds the core; said core comprising statin and/or a pharmaceutically acceptable salt and/or ester thereof and at least one release controlling agent, and said coating characterized by at least one of the a. pH dependent coating film, preferably an enteric coating; b. a combination of at least one water soluble polymer and at least one water insoluble polymer; c. a combination of at least one swellable polymer and at least one water insoluble polymer; d. a combination of at least a water soluble pore forming agent and at least one water insoluble polymer; e. at least one swellable gel forming polymer; f. at least one erodible polymer; g. a combination of at least one pH dependent polymer and at least one water insoluble polymer; h. a two-layer coating comprising a rupturable outer layer and swellable inner layer; characterized in that the in vivo blood plasma concentration of statin is substantially zero for at least about two hours after oral administration.

According to preferred embodiments of the present invention, there is provided a delayed onset controlled release formulation that releases a statin and/or a pharmaceutically acceptable salt and/or ester thereof in the lower gastrointestinal tract of a subject, characterized in that the in vivo blood plasma concentration of said statin and/or a pharmaceutically acceptable salt and/or ester thereof is substantially zero for at least about one hour after oral administration and is controlled by the lag time, providing an increased bioavailability of a statin and/or active forms of said statin, as measured by AUC relative to that resulting from the administration of an equivalent dose of the conventional immediate release formulations.

According to preferred embodiments of the present invention, there is provided a delayed onset controlled release formulation that releases a statin and/or a pharmaceutically acceptable salt and/or ester thereof in the lower gastrointestinal tract of a subject, characterized in that the in vivo blood plasma concentration of said statin and/or a pharmaceutically acceptable salt and/or ester thereof is substantially zero for at least about one hour after oral administration and is controlled by the lag time, providing a controlled absorption of said statin and/or a pharmaceutically acceptable salt and/or ester thereof and/or related active forms, providing an increased bioavailability of a statin and/or active forms of said statin, as measured by AUC, relative to that resulting from the administration of an equivalent dose of the conventional immediate release formulations

Preferably said in vivo blood plasma concentration is extended at least 24 hours.

According to preferred embodiments of the present invention, there is provided a method for providing enhanced bioavailability of statin and/or a pharmaceutically acceptable salts and/or esters thereof and/or its related metabolite in a subject, comprising: administering orally to the subject a controlled release formulation excluding delayed burst release and delayed immediate or fast release characterized in that the in vivo blood plasma concentration of said statin is substantially zero for at least about one hour after oral administration.

Preferably said formulation provides a controlled release after at least two hours resulting in dispersion mainly through the colon of the active ingredient into the blood stream as a result of colon absorption over a period of at least 24 hours.

According to preferred embodiments of the present invention, there is provided a controlled release method for providing an increased amount of a statin and/or active forms of said statin circulating in the blood of a subject, relative to that resulting from the administration of an equivalent dose of a conventional immediate release formulation to the subject, wherein said method excludes delayed burst release and delayed immediate or fast release.

Preferably said formulation comprises a dose of statin and/or a pharmaceutically acceptable salt and/or ester thereof from about 20% to about 60% of a dose of a conventional immediate release formulation.

In one embodiment, the in vivo blood plasma concentration of the statin and/or a pharmaceutically acceptable salt and/or ester thereof is controlled by a lag time, providing a controlled absorption of the statin and/or a pharmaceutically acceptable salt and/or ester thereof and/or related active forms. In one specific embodiment, the formulations of the present invention are characterized in that the in vivo blood plasma concentration of the statin or a pharmaceutically acceptable salt or ester thereof in the subject is substantially zero for at least about 1.5 hours after oral administration of the formulation. In another specific embodiment, the formulations of the present invention are characterized in that the in vivo blood plasma concentration of the statin or a pharmaceutically acceptable salt or ester thereof in the subject is substantially zero for at least about two hours after oral administration of the formulation. In another specific embodiment, the in vivo blood plasma concentration of the statin or a pharmaceutically acceptable salt or ester thereof in the subject is substantially zero for at least about three hours after oral administration of the formulation. In yet another specific embodiment, the in vivo blood plasma concentration of the statin or a pharmaceutically acceptable salt or ester thereof in the subject is substantially zero for at least about four hours after oral administration of the formulation. The term “substantially zero”, as used herein, means that the statin is either not detected in the blood, or only minor amounts of the statin are detected in the blood.

According to one embodiment, the delayed onset controlled release formulation of the present invention provides an increased amount of a statin, a pharmaceutically acceptable salt or ester thereof, or an active form thereof to the circulation of a subject, compared to a substantially similar dose of a conventional immediate release formulation of the stain. As used herein, the term “substantially similar dose” means a dose which is either equivalent or is substantially similar, for example a difference of not more than about 25%. The term “increased amount” means that administration of the formulations of the present invention result in higher blood levels of the statins or their active metabolites (e.g., 10% higher, 20% higher, 50% higher 100% higher, 200% higher, 500% higher etc.), as compared with blood levels achieved by administration of conventional statin formulations. The levels of the statins can be measured by determining the plasma concentration of the statins as a function of time following administration of the formulation, as known to a person of skill in the art. As demonstrated herein, administration of several simvastatin and pitavastatin formulations according to the present invention to subjects resulted in blood levels that were significantly higher than the blood levels achieved after administration of conventional formulations of these statins. Further, importantly, the blood levels were maintained for significantly longer time periods as compared with the conventional formulation. For example, blood levels can be maintained for at least about 6 hours, preferably for about 8 hours, about 10 hours, about 12 hours and most preferably for about 24 hours after the onset of release occurs.

According to an alternative embodiment, the delayed onset controlled release formulation. of the present invention provides enhanced bioavailability of a statin, a pharmaceutically acceptable salt or ester thereof, or an active form thereof in a subject, compared to a substantially similar dose of an immediate release formulation of the stain. The term “enhanced bioavailability” means that administration of the formulations of the present invention results in higher bioavailability of the statins or their active metabolites (e.g., 10% higher, 20% higher, 50% higher 100% higher, 200% higher, 500% higher etc.), as compared with the bioavailability achieved by administration of conventional statin formulations. Bioavailability can be measured for example by comparing the AUC values obtained after administration of the formulations, as known to a person of skill in the art. As demonstrated herein, administration of several simvastatin and pitavastatin formulations according to the present invention to subjects resulted in AUC values that were more than two fold higher than the AUC values obtained after administration of conventional formulations of these statins, Further, the AUC values were maintained for significantly longer time periods as compared with the conventional formulation, for example for at least about 6 hours, preferably for about 8 hours, about 10 hours, about 12 hours and most preferably for about 24 hours after the onset of release occurs.

According to yet another alternative embodiment, the delayed onset controlled release formulation of the present invention provides a therapeutically effective amount of a statin, a pharmaceutically acceptable salt or ester thereof, or an active form thereof into the circulation of a subject. The term “therapeutically effective amount” refers to an amount of the statin which will result in a therapeutic effect of the disease or condition being treated, for example high blood cholesterol.

The present invention represents an improvement over WO 2004/021972 to Biovail, as the Biovail application seeks to reduce the concentration of statins in the blood circulation. In contrast, the present invention provides an increased concentration of statins or active forms thereof in the blood circulation relative to the dose administered, thus resulting in the administration of relatively lower dose of a statin or active forms thereof in the formulation administered to the subject (patient), thereby potentially reducing side effects by decreasing the total dose of statin to which the body of the subject is exposed.

As explained above, the statins are a class of compounds which contain a moiety that can exist as either a 3-hydroxy lactone ring or as the corresponding open ring dihydroxy acid. Typically, the statins can be administered as the inactive lactone prodrugs that must be hydrolyzed in the plasma and liver to the beta-hydroxy acid form for pharmacological activity. In accordance with the present invention, the delayed burst release formulations described herein are capable of providing a therapeutically effective amount of the hydroxy acid metabolite of a statin or a pharmaceutically acceptable salt or ester thereof into the circulation of a subject.

According to other preferred embodiments of the present invention, there is provided a formulation for administering a statin to a subject, featuring a relatively lower dose of said statin, By “relatively lower dose” it is meant a dose that provides at least the same or similar pharmaceutical and/or therapeutic effect (if not a greater effect) as a conventional dose of a statin, while featuring a lower amount of statin than the conventional dose of the statin. It should be noted that a similar principle may optionally be applied for dosage forms featuring a plurality of different statins.

The core of the formulations of the present invention contains a statin, Which is preferably selected from simvastatin, lovastatin, mevastatin, pravastatin, fluvastatin, atorvastatin, cerivastatin and pitavastatin or pharmaceutically acceptable salts, esters, metabolites, hydrates, polymorphs, or crystals thereof. According to one currently preferred embodiment the statin is simvastatin. According to another currently preferred embodiment the statin is pitavastatin. According to other preferred embodiments the statin is lovastatin or atorvastatin.

The term “statin” as used herein includes also pharmaceutically acceptable salts, esters, metabolites, hydrates, polymorphs, or crystals thereof, and includes both statins in the lactone form or in the corresponding open dihydroxy acid.

The term “simvastatin” includes simvastatin and pharmaceutically acceptable salts, esters, metabolites, hydrates, polymorphs, or crystals thereof, in the lactone form or in the corresponding open dihydroxy acid, as disclosed for example in U.S. Pat. No. 4,444,784, which is hereby incorporated by reference in its entirety as if fully set forth herein.

The term “lovastatin” includes lovastatin and pharmaceutically acceptable salts, esters, metabolites, hydrates, polymorphs, or crystals thereof, in the lactone form or in the corresponding open dihydroxy acid, as disclosed for example in U.S. Pat. No. 4,231,938, which is hereby incorporated by reference in its entirety as if fully set forth herein.

The term “mevastatin” includes mevastatin and pharmaceutically acceptable salts, esters, metabolites, hydrates, polymorphs, or crystals thereof, in the lactone form or in the corresponding open dihydroxy acid, as disclosed for example in U.S. Pat. No. 3,671,523, which is hereby incorporated by reference in its entirety as if fully set forth herein.

The term “pravastatin” includes pravastatin and pharmaceutically acceptable salts, esters, metabolites, hydrates, polymorphs, or crystals thereof, in the lactone form or in the corresponding open dihydroxy acid, as disclosed for example in U.S. Pat. No. 4,346,227, which is hereby incorporated by reference in its entirety as if fully set forth herein.

The term “fluvastatin” includes fluvastatin and pharmaceutically acceptable salts, esters, metabolites, hydrates, polymorphs, or crystals thereof, in the lactone form or in the corresponding open dihydroxy acid, as disclosed for example in U.S. Pat. No. 5,354,772, which is hereby incorporated by reference in its entirety as if fully set forth herein.

The term “atorvastatin” includes atorvastatin and pharmaceutically acceptable salts, esters, metabolites, hydrates, polymorphs, or crystals thereof, in the lactone form or in the corresponding open dihydroxy acid, as disclosed for example in U.S. Pat. No. 5,273,995, which is hereby incorporated by reference in its entirety as if fully set forth herein.

The term “rivastatin” includes rivastatin and pharmaceutically acceptable salts, esters, metabolites, hydrates, polymorphs, or crystals thereof, in the lactone form or in the corresponding open dihydroxy acid, as disclosed for example in U.S. Pat. No. 5,177,080, which is hereby incorporated by reference in its entirety as if fully set forth herein.

The term “pitavastatin” (“nisvastatin”) includes pitavastatin and pharmaceutically acceptable salts, esters, metabolites, hydrates, polymorphs, or crystals thereof, in the lactone form or in the corresponding open dihydroxy acid, as disclosed for example in U.S. Pat. No. 5,011,930, U.S. Pat. No. 5,872,130, U.S. Pat. No. 5,856,336, which are hereby incorporated by reference in their entirety as if fully set forth herein.

As used herein, the term “active form” refers to any form of a molecule that can function as an HMG-CoA reductase inhibitor including the active ingredient administered and any active derivative resulting from metabolism or otherwise obtained from the parent molecule that can act as an HMG-CoA reductase. For example in the case of simavastatin marketed under the tradename ZOCOR® the known active forms include α-hydroxyacid of simvastatin and its 6β-hydroxy, 6β-hydroxymethyl, and 6β-exomethylene derivatives. The term “metabolite”, as used herein, includes any active form of the statin as described herein.

Suitable pharmaceutically acceptable salts include but are not limited to inorganic salts such as, for example, sodium, potassium, ammonium, calcium, and the like.

The doses of the statins to be used in the formulations of the present invention can be determined by a person of skill in the art, and will vary depending on the statin being used, the patient, and the condition being treated. Typical known therapeutic doses for each of the statins can be used as a guide to determine the appropriate dose to be used herein. As mentioned above, the formulations of the present invention preferably contain a reduced dose of the statin, as compared with the corresponding conventional formulation, preferably up to about 60% of the conventional dose for each statin.

### BRIEF DESCRIPTION OF THE DRAWINGS

The invention is herein described, by way of example only, with reference to the accompanying drawings, wherein:

FIG. 1 shows the in-vitro dissolution profile for tablets featuring different amounts of binder/filler according to the present invention;

FIG. 2 shows the in-vitro dissolution profile for other formulations for tablets featuring different amounts of binder/filler according to the present invention;

FIG. 3 shows the in-vitro dissolution profile for a tablet featuring an enteric coating according to the present invention;

FIG. 4 shows the in-vitro dissolution profile for a tablet featuring a dry coating according to the present invention; and

FIG. 5 shows the in-vitro dissolution profile for a tablet featuring a coating having a combination of water insoluble polymer and hydrophilic but water insoluble particles according to the present invention.

### DESCRIPTION OF THE PREFERRED EMBODIMENTS

The present invention provides a delayed onset controlled release formulation for controlled absorption of a statin, adapted so as to provide a time-delayed, controlled release in the colon or small intestine. The formulation supports a lag time between oral administration and release of the active ingredient, providing higher bioavailability and lower dosage as compared to the currently used formulation. The term “statin” includes also pharmaceutically acceptable salts or esters thereof.

The term “controlled release” includes any formulation of delayed slow release, delayed sustained release, delayed prolonged release, and delayed extended release.

The delivery system of the present invention provides a modified formulation comprising a statin for delayed onset controlled delivery of the active ingredient to the gastrointestinal tract. The delivery system comprises a drug containing core surrounded by a coating that limits the access of liquid to the core thereby controlling the release of the drug from the core to the GI tract.

The formulation is optionally in the form of a coated tablet. Alternatively, the formulation may be in the form of a pellet, microparticles, agglomerate, capsule or any other solid dosage form.

The core contains a statin, which is selected from simvastatin, lovastatin, mevastatin, pravastatin, fluvastatin, atorvastatin and cerivastatin, pitavastatin or pharmaceutically acceptable salts thereof. According to one currently more preferred embodiment the statin is simvastatin; according to other preferred embodiments the statin is lovastatin or atorvastatin.

The combination of the selected materials for the core and outer layer, and the relative concentrations thereof, as well as the thickness of the core matrix and outer layer, determine both the lag time, which is the time, post administration, when the release starts, as well as the rate of release of the drug.

### Controlled Release Core

An optional but preferred embodiment of a formulation according to the present invention comprises a controlled release core which is preferably a slow release core.

The core comprises the active ingredient and one or more other ingredients which provided the delayed onset, control release profile, which is preferably a slow release profile.

According to an optional but preferred embodiment of the present invention, there is provided a swellable core, featuring at least one swellable polymer. The swellable polymers are hydrophilic polymers. Suitable swellable, hydrogel-forming polymers include, but are not limited to, poly (hydrox alkyl methacrylate) having a molecular weight of from 30,000 to 5,000.000; kappa-carrageenan; polyvinylpyrrolidone having a molecular weight of from 10,000 to 360,000; anionic and cationic hydrogels; polyelectrolyte complexes; poly(vinyl alcohol) having low amounts of acetate, cross-linked with glyoxal, formaldehyde, or glutaraldehyde and having a degree of polymerization from 200 to 30,000; a mixture including methyl cellulose, cross-linked agar and carboxymethyl cellulose; a water-insoluble, water-swellable copolymer produced by forming a dispersion of finely divided maleic anhydride with styrene, ethylene, propylene, butylene or isobutylene; water-swellable polymers of N-vinyl lactams; polysaccharide, water swellable gums, high viscosity hydroxylpropylmethyl cellulose and/or mixtures of any of the foregoing.

The swellable core preferably also features a wicking controlling agent, such as silicon dioxide for example. Wicking agents such as those materials described herein as disintegrants (e.g. microcrystalline cellulose) may be included if necessary to enhance the speed of water uptake. Other materials suitable for acting as wicking agents include, but are not limited to, kaolin, titanium dioxide, fumed silicon dioxide, alumina, niacinamide, sodium lauryl sulfate, low molecular weight polyvinyl pyrrolidone, m-pyrol, bentonite, magnesium aluminum silicate, polyester, polyethylene, mixtures thereof, and the like.

According to an optional but preferred embodiment of the present invention, there is provided a delayed onset, slow release core, featuring at least one release controlling agent.

The release controlling agent may comprise suitable hydrophilic gelling polymers including but not limited to cellulosic polymers, such as methylcellulose, carboxymethylcellulose, hydroxypropylcellulose, hydroxypropylmethylcellulose, hydroxyethylcellulose, and the like; vinyl polymers, such as polyvinylpyrrolidone, polyvinyl alcohol, and the like; acrylic polymers and copolymers, such as acrylic acid polymer, methacrylic acid copolymers, ethyl acrylate-methyl methacrylate copolymers, natural and synthetic gums, such as guar gum, arabic gum, xanthan gum, gelatin, collagen, proteins, polysaccharides, such as pectin, pectic acid, alginic acid, sodium alginate, polyaminoacids, polyalcohols, polyglycols; and the like; and mixtures thereof. The preferred swellable polymeric coating layer comprises hydroxypropylmethylcellulose.

The polysaccharide may optionally be a cross-linked polysaccharide. Preferably, the cross-linked polysaccharide is selected from the group consisting of insoluble metal salts or cross-linked derivatives of alginate, pectin, xantham gum, guar gum, tragacanth gum, and locust bean gum, carrageenan, metal salts thereof, and covalently cross-linked derivatives thereof.

The cellulosic polymer may optionally be a modified cellulose. Preferably, the modified cellulose is selected from the group consisting of cross-linked derivatives of hydroxypropylcellulose, hydroxypropylmethylcellulose, hydroxyethylcellulose, methylcellulose, carboxymethylcellulose, and metal salts of carboxymethylcellulose.

According to other optional but preferred embodiments of the present invention, the core is a delayed onset, slow release core in which the time and rate of release are controlled through the amount of release controlling agent. The range of amounts of the release controlling agent preferably are from about 5 to about 20% weight by weight of the core weight.

Preferably, the core further comprises at least one of an absorption enhanced, a binder, a hardness enhancing agent, optionally a disintegrant and another excipient. More preferably, the binder is selected from the group consisting of Povidone (PVP: polyvinyl pyrrolidone), low molecular weight HPC (hydroxypropyl cellulose), low molecular weight HPMC (hydroxypropyl methylcellulose), low molecular weight carboxy methyl cellulose, ethylcellulose, gelatin polyethylene oxide, acacia, dextrin, magnesium aluminum silicate, starch, and polymethacrylates. Optionally and preferably, the core also includes a stabilizer. More preferably, the stabilizer comprises at least one of butyl hydroxyanisole, ascorbic acid and citric acid.

More preferably, the disintegrant is selected from the group consisting of croscarmellose sodium, crospovidone (cross-linked polyvinyl pyrolidone) sodium carboxymethyl starch (sodium starch glycolate), cross-linked sodium carboxymethyl cellulose (Croscarmellose), pregelatinized starch (starch 1500), microcrystalline starch, water insoluble starch, calcium carboxymethyl cellulose, magnesium aluminum silicate and a combination thereof. More preferably, the disintegrating agent is croscarmellose sodium.

The mechanism of disintegration is based on swelling, wicking, and deformation of the disintegrants. Some commercial superdisintegrants for use in the present invention include but are not limited to Ac-Di-Sol, Primojel, Explotab, and Crospovidone.

The core of the present invention optionally and preferably includes a wicking agent in addition to or as an alternative to a disintegrant; wicking agents are described above.

Alternatively or additionally, the core further comprises a filler. Preferably, the filler is selected from the group consisting of microcrystalline cellulose, starch, lactitol, lactose, a suitable inorganic calcium salt, sucrose, or a combination thereof. More preferably the filler is lactose monohydrate.

According to optional embodiments of the present invention, the core comprises a release controlling agent, preferably present in a range of from about 5% to about 20% of the core weight by weight, and a filler, but does not include a disintegrating agent. The filler is preferably present in an amount such that a ratio of filler to release controlling agent is in a range of from about 10% to about 85% (W/W) relatively to the total weight of core.

More preferably, the core further includes a chelating agent to increase chelation of trace quantities of metals thereby helping in preventing the loss of a statin such as Simvastatin by oxidation. Most preferably, the chelating agent is citric acid.

According to preferred embodiments of the present invention, the core further comprises a synergistic agent (sequestrate). Preferably, the sequestrate is selected from the group consisting of citric acid and ascorbic acid.

Without wishing to be limited by a single hypothesis, chelating agents and sequestrates may optionally be differentiated as follows. A chelating agent, such as (preferably) citric acid is intended to help in chelation of trace quantities of metals thereby assisting to prevent the loss of the active ingredient(s), such as a statin such as Simvastatin for example, by oxidation.

A sequestrate such as (preferably) ascorbic acid, optionally and preferably has several hydroxyl and/or carboxylic acid groups, which can provide a supply of hydrogen for regeneration of the inactivated Butyl hydroxyanisole (BHA) antioxidant free radical. A sequestrate therefore preferably acts as a supplier of hydrogen for rejuvenation of the primary antioxidant.

According to preferred embodiments of the present invention, the core further comprises an antioxidant. Preferably, the antioxidant is selected from the group consisting of 4,4 (2,3 dimethyl tetramethylene dipyrochatechol), Tocopherol-rich extract (natural vitamin E), α-tocopherol (synthetic Vitamin E), β-tocopherol, γ-tocopherol, δ-tocopherol, Butylhydroxinon, Butyl hydroxyanisole (BHA), Butyl hydroxytoluene (BHT), Propyl Gallate, Octyl gallate, Dodecyl Gallate, Tertiary butylhydroquinone (TBHQ), Fumaric acid, Malic acid, Ascorbic acid (Vitamin C), Sodium ascorbate, Calcium ascorbate, Potassium ascorbate, Ascorbyl palmitate, Ascorbyl stearate, Citric acid, Sodium lactate, Potassium lactate, Calcium lactate, Magnesium lactate, Anoxomer, Erythorbic acid, Sodium erythorbate, Erythorbin acid, Sodium erythorbin, Ethoxyquin, Glycine, Gum guaiac, Sodium citrates (monosodium citrate, disodium citrate, trisodium citrate), Potassium citrates (monopotassium citrate, tripotassium citrate), Lecithin, Polyphosphate, Tartaric acid, Sodium tartrates (monosodium tartrate, disodium tartrate), Potassium tartrates (monopotassium tartrate, dipotassium tartrate), Sodium potassium tartrate, Phosphoric acid, Sodium phosphates (monosodium phosphate, disodium phosphate, trisodium phosphate), Potassium phosphates (monopotassium phosphate, dipotassium phosphate, tripotassium phosphate), Calcium disodium ethylene diamine tetra-acetate (Calcium disodium EDTA), Lactic acid, Trihydroxy butyrophenone and Thiodipropionic acid.

More preferably, the core further comprises ascorbic acid, which has several hydroxyl and/or carboxylic acid groups, and is able to provide a supply of hydrogen for regeneration of the primary antioxidant, exerting a synergistic effect on the inactivated antioxidant free radical. Most preferably, the primary antioxidant is BHA. According to preferred embodiments of the present invention, the core further comprises a chelating agent. Preferably, the chelating agent is selected from the group consisting of Antioxidants, Dipotassium edentate, Disodium edentate, Edetate calcium disodium, Edetic acid, Fumaric acid, Malic acid, Maltol, Sodium edentate, Trisodium edetate. Also alternatively or additionally, the core further comprises a flow regulating agent. Preferably, the flow regulating agent includes at least one of colloidal silicon dioxide and aluminum silicate. Most preferably, the flow regulating agent is colloidal silicon dioxide. Preferably, the core further comprises a lubricant. More preferably, the lubricant is selected from the group consisting of stearate salts; stearic acid, corola oil, glyceryl palmitostearate, hydrogenated vegetable oil, magnesium oxide, mineral oil, poloxamer, polyethylene glycole, polyvinyl alcohol, sodium benzoate, talc, sodium stearyl fumarate, compritol (glycerol behenate), and sodium lauryl sulfate (SLS) or a combination thereof. Most preferably, the lubricant is magnesium stearate.

A preferred embodiment of the formulation according to the present invention preferably features a core which contains a hydrophilic, swellable, hydrogel-forming material, covered by a coating which includes a water insoluble polymer and hydrophilic water permeable agent, through which water enters the core. The swellable hydrogel-forming material in the core then swells and bursts the coating, after which the core more preferably disintegrates slowly or otherwise releases the active ingredient. Another optional but preferred embodiment relates to a release-controlling core with an slow-erodible dry coating.

Release of the active agent of the present formulation preferably occurs within about 8 hours of oral administration, with a slightly longer delay occurring with the enteric coated embodiment.

In one embodiment the controlled release formulation is based on a combination of a release-controlling core and a pH dependent coating film more preferably an enteric coating. The core can be based on either a swellable hydrogel-forming formulation or swellable, hydrogel-forming, slow disintegrating formulation, but in any case it is preferably a slow disintegrating formulation.

Such a formulation can prevent release of the active ingredient in the stomach and even in the upper GI tract for a predetermined period of time, for example up to about 2 hours, more preferably up to about 3 to 4 hours, after which the release can take place in a controlled manner. The core according to such an embodiment may comprise the active ingredient, optionally a disintegrant (as noted above, according to optional but preferred embodiments of the present invention, the core specifically does not feature a disintegrant) and a release-controlling agent which is preferably a hydrophilic, swellable, hydrogel-forming polymer, in which the core is preferably formed as a compressed tablet. More preferably, the core is in the form of one of a tablet, pellets, microparticles, agglomerate, and capsule.

### Outer Coating

The core is preferably coated with a rate-controlling coating according to the present invention. The coating is optionally and preferably selected from the group consisting of a pH dependent coating film (featuring a pH dependent polymer), preferably an enteric coating; a combination of at least one water insoluble polymer and at least one water insoluble hydrophilic particles; a combination of two or more water insoluble polymers; a combination of at least one swellable polymer and at least one water insoluble polymer; a combination of at least a water soluble pore forming agent and at least one water insoluble polymer; a dry coating; a flexible but non-soluble coating (the latter is preferably combined with a swellable core); a swellable coating, which may optionally be a dry coating featuring a gel forming material; and a water permeable coating.

According to this embodiment of the present invention, the pH dependent polymer of the outer coating is selected from the group consisting of a hydroxypropylmethyl cellulose phthalate, polyvinyl acetate phthalate, cellulose acetate phthalate, hydroxypropylmethyl cellulose acetate succinate, poly(methacrylic acid, methyl methacrylate)1:1 and poly(methacrylic acid, ethyl acrylate)1:1, alginic acid, and sodium alginate. A suitable enteric coating can be from Eudragit™ polymers series (available from Rohm Pharma) which are polymeric lacquer substances based on acrylates and/or methacrylates. Suitable polymers which are slightly permeable to water, and exhibit a pH-dependent permeability include, but are not limited to, Eudragit™ L, and Eudragit™ S. Eudragit™ L is an anionic polymer synthesized from methacrylic acid and methacrylic acid methyl ester. It is insoluble in acids and pure water. It becomes soluble in neutral to weakly alkaline conditions. The permeability of Eudragit™ L is pH dependent. Above pH 5.0, the polymer becomes increasingly permeable.

An illustrative, non-limiting example of such a formulation is as follows. The formulation optionally and preferably comprises a pH dependent film coat, the polymeric material comprises methacrylic acid co-polymers, ammonio methacrylate co-polymers, or a mixture thereof. Methacrylic acid co-polymers such as Eudragit™ S and Eudragit™ L (Rohm Pharma) are suitable for use in the delayed onset, modified, release formulations of the present invention, these polymers are gastro-resistant and entero-soluble polymers, providing a delay in onset of the release depending on the pH, the type of the polymer (Eudragit L or Eudragit S) and the thickness of the film coat.

The films of Methacrylic acid co-polymers such as Eudrait™ S and Eudragit™ L are insoluble in pure water and diluted acids. They dissolve at higher pH values, depending on their content of carboxylic acid. Eudragit™ S and Eudragit™ L can be used as single components in the coating of the formulation of the present invention or in combination in any ratio. By using a combination of the polymers, the polymeric material may exhibit a solubility at a pH between the pHs at which Eudragit™ L and Eudragit™ S are separately soluble.

Optionally, the outer coating further comprises a plasticizer. More preferably, the plasticizer includes at least one of dibutyl sebacate, polyethylene glycol and polypropylene glycol, dibutyl phthalate, diethyl phthalate, triethyl citrate, tributyl citrate, acetylated monoglyceride, acetyl tributyl citrate, triacetin, dimethyl phthalate, benzyl benzoate, butyl and/or glycol esters of fatty acids, refined mineral oils, oleic acid, castor oil, corn oil, camphor, glycerol and sorbitol or a combination thereof.

In another embodiment according to the present invention the delayed onset, controlled release formulation may comprise a release controlling core formulation and an outer coating, optionally comprising a combination of a water soluble polymer and/or a water swellable hydrophilic polymer and a water insoluble polymer. In this manner, where the film coating formulation features a combination of at least a water soluble polymer and at least a water insoluble polymer, it is possible to provide a delay time prior to the release of the active material, depending on the relative content (weight fraction) of the water soluble polymer in the outer coating, the thickness of the outer film coat, and the nature of the polymers present in the outer film coat. Without wishing to be limited by a single hypothesis, upon exposure of the formulation to the gastrointestinal fluids, the water soluble polymer starts to dissolve, leaving channels that allow penetration of the gastrointestinal fluids into the core, which may eventually lead to a slow disintegration of the core and thus a slow release of the active material.

A non-limiting, illustrative example according to this embodiment may be based on a core which can be formulated as described above for the previous embodiment, and an outer coating comprising a totally water soluble polymer and a water insoluble polymer. Without wishing to be limited by a single hypothesis, coating disintegration may be due to increased osmotic pressure in the core. Suitable water-soluble polymers include, but are not limited to, polyvinyl alcohol, polyvinylpyrrolidone (PVP), copolyvidone (a (6:4) copolymer of a chain-structured vinyl pyrrolidone and vinyl acetate), methylcellulose, hydroxypropylcellulose, hydroxypropylmethyl cellulose, polyethylene glycol, carboxymethyl cellulose (sodium salt), hydroxyethyl cellulose, a water soluble gum, polysaccharide and/or mixtures thereof.

Suitable water insoluble polymers of the outer coating are selected from the group consisting of a polymethylaminoethylacrylate/ethylmethacrylate copolymer, the copolymer being based on acrylic and methacrylic acid esters with a low content of quaternary ammonium groups, wherein the molar ratio of the ammonium groups to the remaining neutral (meth)acrylic acid esters is approximately 1:20, the polymer corresponding to USP/NF “Ammonio Methacrylate Copolymer Type A”, an ethylmethacrylate/chlorotrimethylammoniumethyl methacrylate copolymer, the copolymer based on acrylic and methacrylic acid esters with a low content of quaternary ammonium groups wherein the molar ratio of the ammonium groups to the remaining neutral (meth)acrylic acid esters is 1:40, the polymer corresponding to USP/NF “Ammonio Methacrylate Copolymer Type B”, a dimethylaminoethylmethacrylate/methylmethacrylate and butylmethacrylate copolymer, a copolymer based on neutral methacrylic acid esters and dimethylaminoethyl methacrylate esters wherein the polymer is cationic in the presence of acids, an ethylacrylate and methylacrylate/ethylmethacrylate and methyl methylacrylate copolymer, the copolymer being a neutral copolymer based on neutral methacrylic acid and acrylic acid esters, ethylcellulose, shellac, zein, and waxes, paraffin, cellulose acetate, cellulose propionate, cellulose acetate propionate, cellulose acetate butyrate, cellulose acetate phthalate, cellulose triacetate, poly (methyl methacrylate), poly(ethylmethacrylate), poly (butyl methacrylate), poly (isobutyl methacrylate), and poly (hexyl methacrylate), poly (isodecyl methacrylate), poly(lauryl methacrylate), poly (phenyl methacrylate), poly (methylacrylate), poly (isopropyl acrylate), poly (isobutyl acrylate) poly(octadecyl acrylate), poly (ethylene), poly (ethylene) low density, poly(ethylene) high density, poly (ethylene oxide), poly (ethyleneterephthalate), poly (vinyl isobutyl ether), poly (vinyl acetate), poly(vinyl chloride) and polyurethane, and/or mixtures thereof. More preferably, the water insoluble polymer is ethylcellulose.

Optionally, the formulation may further comprise an enteric coating disposed on the outer coating. Enteric coatings may optionally be selected from the examples described previously.

Another example according to this embodiment may be based on a swellable hydrogel-forming, slowly disintegrating core and an outer coating comprising a combination of a water swellable hydrophilic polymer and a water insoluble film-forming polymer. The swellable polymer may be a gel-forming polymer. This enables the swellable polymer participating in the outer film coat composition to be free of the requirement to fully dissolve. Since the swelling process of the swellable polymer in the outer film coat composition controls the diffusion process of the GI fluid through the film coat into the core, without wishing to be limited by a single hypothesis it is expected that it will be the only key factor for controlling the lag time. Another factor controlling the lag time is the thickness of the outer film coat.

Suitable swellable polymers typically interact with water and/or gastrointestinal fluids, which causes them to swell or expand to an equilibrium state. Acceptable polymers exhibit the ability to swell in water and/or gastrointestinal fluids, retaining a significant portion of such imbibed fluids within their polymeric structure. The polymers may swell or expand, usually exhibiting a 2- to 50-fold volume increase. The polymers can be non-cross-linked or cross-linked. The swellable hydrophilic polymer is responsible for introducing the gastrointestinal fluids into the core, leading to swelling of the core and eventually release of the active material. The swellable polymers are hydrophilic polymers. Suitable polymers include, but are not limited to, poly(hydrox alkyl methacrylate) having a molecular weight of from 30,000 to 5,000.000; kappa-carrageenan; polyvinylpyrrolidone having a molecular weight of from 10,000 to 360,000; anionic and cationic hydrogels; polyelectrolyte complexes; poly(vinyl alcohol) having low amounts of acetate, cross-linked with glyoxal, formaldehyde, or glutaraldehyde and having a degree of polymerization from 200 to 30,000; a mixture including methyl cellulose, cross-linked agar and carboxymethyl cellulose; a water-insoluble, water-swellable copolymer produced by forming a dispersion of finely divided maleic anhydride with styrene, ethylene, propylene, butylene or isobutylene; water-swellable polymers of N-vinyl lactams; polysaccharide, water swellable gums, high viscosity of hydroxylpropylmethyl cellulose and/or mixtures of any of the foregoing. The outer film coat according to this embodiment, can also include a material that improves the processing of the polymers. Such materials are generally referred to as plasticizers and include, for example, adipates, azelates, benzoates, citrates. isoebucates, phthalates, sebacates, stearates and glycols. Representative plasticizers include acetylated monoglycerides, butyl phthalyl butyl glycolate, dibutyl tartrate, diethyl phthalate, dimethyl phthalate, ethyl phthalyl ethyl glycolate, glycerin, ethylene glycol, propylene glycol, triacetin citrate, triacetin, tripropinoin, diacetin, dibutyl phthalate, acetyl monoglyceride, polyethylene glycols, castor oil, triethyl citrate, polyhydric alcohols, acetate esters, glycerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-1-octyl phthalate, di-1-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, glyceryl monocaprylate, and glyceryl monocaprate. In one embodiment, the plasticizer is dibutyl sebacate. The amount of plasticizer used in the polymeric material typically ranges from about 10% to about 50%, for example, about 10, 20, 30, 40 or 50%, based on the weight of the dry polymer.

Optionally, the formulation may comprise an enteric coating disposed on the outer coating.

In another embodiment, the outer film coat comprises one or more water-insoluble film-forming polymers and one or more water-soluble pore-forming compounds. Suitable water-soluble pore-forming compounds include, but are not limited to, saccharose, sodium chloride, potassium chloride, polyvinylpyrrolidone, and/or polyethyleneglycol, water soluble organic acids, sugars and sugar alcohol. The pore-forming compounds may be uniformly or randomly distributed throughout the water insoluble polymer. Typically, the pore-forming compounds comprise about 1 part to about 35 parts for each about 1 to about 10 parts of the water insoluble polymers. The amount and particle size of pore-forming agent in the film coat, and the thickness of the outer film coat are expected to be the main parameters controlling the lag time. Optionally, the formulation may comprise an enteric coating disposed on the outer coating.

According to other preferred embodiments of the present invention, there is provided a combination of swellable core and coating which includes a water insoluble but flexible polymer and hydrophilic water permeable hydrophilic particles, through which water enters the core. The swellable material in the core then swells and stretches the flexible component of coating leading to increase of coating permeability and delayed sustain release of active material.

Without wishing to be limited by a single hypothesis, this type of coating provides delayed onset diffusion controlled release. Suitable polymers for comprising the water insoluble flexible polymer include but are not limited to Methacrylic acid co-polymers such as Eudragit E or Eudragit NE 30 D.

The water insoluble hydrophilic particles optionally comprise a polymer that forms a hydrogel upon contact with liquid. Preferably, the polymer is selected from the group consisting of poly(hydrox alkyl methacrylate); kappa-carrageenan; polyvinylpyrrolidone; anionic and cationic hydrogels; polyelectrolyte complexes; poly(vinyl alcohol) having low amounts of acetate, cross-linked with glyoxal, formaldehyde, or glutaraldehyde; a mixture comprising methyl cellulose, cross-linked agar and carboxymethyl cellulose; a water-insoluble, water-swellable copolymer produced by forming a dispersion of finely divided maleic anhydride with styrene, ethylene, propylene, butylene or isobutylene; water-swellable polymers of N-vinyl lactams; polysaccharide, water swellable gums, high viscosity hydroxylpropylmethyl cellulose and/or mixtures thereof.

Optionally and more preferably, the water insoluble particles comprise a non-gel-forming hydrophilic polymer selected from the group consisting of cross-linked polysaccharide, water insoluble starch, microcrystalline cellulose, water insoluble cross-linked peptide, water insoluble cross-linked protein, water insoluble cross-linked gelatin, water insoluble cross-linked hydrolyzed gelatin, water insoluble cross-linked collagen, modified cellulose, and cross-linked polyacrylic acid. Most preferably, the cross-linked polysaccharide is selected from the group consisting of insoluble metal salts or cross-linked derivatives of alginate, pectin, xantham gum, guar gum, tragacanth gum, and locust bean gum, carrageenan, metal salts thereof, and covalently cross-linked derivatives thereof.

Optionally and more preferably, the modified cellulose is selected from the group consisting of cross-linked derivatives of hydroxypropylcellulose, hydroxypropylmethylcellulose, hydroxyethylcellulose, methylcellulose, carboxymethylcellulose, and metal salts of carboxymethylcellulose.

Particularly preferred examples of such a water insoluble polymer include calcium pectinate or microcrystalline cellulose.

In another embodiment a delayed onset, controlled release formulation based on a dry compress coating process may be considered. Such a dosage form may optionally feature a slowly disintegrating core coated with an erodible composition using a double compress tabletation. Such an erodible composition may be a slow dissolving or slow disintegrating pharmaceutically acceptable excipients such as, but not limited to, slowly water soluble polymers, swellable polymer or a composition comprising a slowly water soluble polymer with a disintegrant or a swellable polymer with disintegrant. Alternatively the coating process can be carried out using a conventional coating process such as spraying of an erodible or swellable polymer. Such a solution may optionally include additional excipients like a disintegrant and talc.

When an erodible polymer is used, the erosion rate of such a coating may determine the lag time, therefore, the type of polymer being used as erodible polymer, may be expected to control the erosion rate of the coating can determine the lag time. Another parameter that can control the lag time is the amount of erodible polymer constituting the thickness of the coating.

When a swellable polymer is used, the coating layer, which typically comprises a hydrophilic gelling polymer or swellable polymer, swells on contact with gastro-intestinal juices to form a continuous film surrounding the core. The coating layer must sufficiently protect the integrity of the core for the desired period of time, without regard to the pH of the medium to which it is subjected. Once the desired, pre-delivery time period has elapsed, the core should be capable of relatively slow disintegration so that the statin is released in a controlled release manner at the predetermined delivery time.

The polymeric coating layer may be comprised of any suitable hydrophilic gelling polymer known to those skilled in the art. For example, suitable hydrophilic gelling polymers include but are not limited to cellulosic polymers, such as methylcellulose, carboxymethylcellulose, hydroxypropylcellulose, hydroxypropylmethylcellulose, hydroxyethylcellulose, and the like; vinyl polymers, such as polyvinylpyrrolidone, polyvinyl alcohol, and the like; acrylic polymers and copolymers, such as acrylic acid polymer, methacrylic acid copolymers, ethyl acrylate-methyl methacrylate copolymers, natural and synthetic gums, such as guar gum, arabic gum, xanthan gum, gelatin, collagen, proteins, polysaccharides, such as pectin, pectic acid, alginic acid, sodium alginate, polyaminoacids, polyalcohols, polyglycols; and the like; and mixtures thereof. The preferred swellable polymeric coating layer comprises hydroxypropylmethylcellulose.

Alternatively, the swellable polymeric coating layer may be comprised of other substances which are capable of becoming freely permeable with exactly defined kinetics following hydration in aqueous fluids. Such substances include, saccharose, sorbitol, mannoses, and jaluronic acid; and the like.

In addition to the foregoing, the swellable polymeric coating layer may also include additional excipients such as lubricants, flow promoting agents, plasticizers, anti-sticking agents, natural and synthetic flavorings and natural and synthetic colorants. Specific examples of additional excipients include polyethylene glycol, polyvinylpyrrolidone, talc, magnesium stearate, glyceryl behenate, stearic acid, and titanium dioxide.

The swellable polymeric coating layer may be applied to the core using conventional film (or spray) coating techniques, double press coating or by the method involving the alternate application of binder and powdered polymeric coating particles.

In certain embodiments, gums for use in the compression coating include, for example and without limitation, heteropolysaccharides such as xanthan gum(s), homopolysaccharides such as locust bean gum, galactans, mannans, vegetable gums such as alginates, gum karaya, pectin, agar, tragacanth, accacia, carrageenan, tragacanth, chitosan, agar, alginic acid, other polysaccharide gums (e.g. hydrocolloids), and mixtures of any of the foregoing. Further examples of specific gums which may be useful in the compression coatings of the invention include but are not limited to acacia catechu, salai guggal, indian bodellum, copaiba gum, asafetida, cambi gum, Enterolobium cyclocarpum, mastic gum, benzoin gum, sandarac, gambier gum, butea frondosa (Flame of Forest Gum), myrrh, konjak mannan, guar gum, welan gum, gellan gum, tara gum, locust bean gum, carageenan gum, glucomannan, galactan gum, sodium alginate, tragacanth, chitosan, xanthan gum, deacetylated xanthan gum, pectin, sodium polypectate, gluten, karaya gum, tamarind gum, ghatti gum, Accaroid/Yacca/Red gum, dammar gum, juniper gum, ester gum, ipil-ipil seed gum, gum talha (acacia seyal), and cultured plant cell gums including those of the plants of the genera: acacia, actinidia, aptenia, carbobrotus, chickorium, cucumis, glycine, hibiscus, hordeum, letuca, lycopersicon, malus, medicago, mesembryanthemum, oryza, panicum, phalaris, phleum, poliathus, polycarbophil, sida, solanum, trifolium, trigonella, Afzelia africana seed gum, Treculia africana gum, detarium gum, cassia gum, carob gum, Prosopis africana gum, Colocassia esulenta gum, Hakea gibbosa gum, khaya gum, scleroglucan, zea, mixtures of any of the foregoing, and the like.

In certain especially preferred embodiments, the compression coating comprises a heteropolysaccharide such as xanthan gum, a homopolysaccharide such as locust bean gum, or a mixture of one or more hetero- and one or more homopolysaccharide(s). Heterodisperse excipients, previously disclosed as a sustained release tablet matrix in U.S. Pat. Nos. 4,994,276, 5,128,143, and 5,135,757, may be utilized in the compression coatings of the present invention. For example, in certain embodiments of the present invention, a gelling agent of both hetero- and homo-polysaccharides which exhibit synergism, e.g., the combination of two or more polysaccharide gums producing a higher viscosity and faster hydration than that which would be expected by either of the gums alone, the resultant gel being faster-forming and more rigid, may be used in the compression coatings of the present invention.

The term “heteropolysaccharide” as used in the present invention is defined as a water-soluble polysaccharide containing two or more kinds of sugar units, the heteropolysaccharide having a branched or helical configuration, and having excellent water-wicking properties and immense thickening properties.

An especially preferred heteropolysaccharide is xanthan gum, which is a high molecular weight (>10.sup.6) heteropolysaccharide. Other preferred heteropolysaccharides include derivatives of xanthan gum, such as deacylated xanthan gum, the carboxymethyl ether, and the propylene glycol ester.

The homopolysaccharide materials used in the present invention that are capable of cross-linking with the heteropolysaccharide include the galactomannans, i.e., polysaccharides that are composed solely of mannose and galactose. A possible mechanism for the interaction between the galactomannan and the heteropolysaccharide involves the interaction between the helical regions of the heteropolysaccharide and the unsubstituted mannose regions of the galactomannan. Galactomannans that have higher proportions of unsubstituted mannose regions have been found to achieve more interaction with the heteropolysaccharide. Hence, locust bean gum, which has a higher ratio of mannose to galactose, is especially preferred as compared to other galactomannans, such as guar and hydroxypropyl guar.

According to an optional but preferred embodiment of the present invention, there is provided a coating comprising an enteric coating. Preferably, the enteric coating comprises Hydroxypropylmethyl cellulose acetate succinate (HPMC AS).

More preferably, HPMC AS is present in an amount ranging from about 25% to about 90% of the enteric coating. Optionally and more preferably, the coating comprises a plasticizer. Most preferably, the plasticizer comprises triethyl citrate. Also optionally and more preferably, the coating comprises a surfactant. Most preferably, the surfactant comprises sodium lauryl sulfate.

According to another embodiment of the present invention, the coating features a water insoluble but water permeable polymer, preferably comprising material selected from the group consisting of cross-linked polysaccharide, water insoluble starch, microcrystalline cellulose, water insoluble cross-linked peptide, water insoluble cross-linked protein, water insoluble cross-linked gelatin, water insoluble cross-linked hydrolyzed gelatin, water insoluble cross-linked collagen, modified cellulose, and cross-linked polyacrylic acid. Most preferably, the cross-linked polysaccharide is selected from the group consisting of insoluble metal salts or cross-linked derivatives of alginate, pectin, xantham gum, guar gum, tragacanth gum, and locust bean gum, carrageenan, metal salts thereof, and covalently cross-linked derivatives thereof.

An additional embodiment comprises a tablet system consisting of a slowly disintegrating core coated with two distinct layers of swelling and rupturable coating layers. The slowly disintegrating core containing statin is coated sequentially with an inner swelling layer containing superdisintegrant and an outer rupturable layer of a brittle polymer. The latter coating layer may include at least one permeation-enhancing agent in order to promote the diffusion of water into the rupturable coating layer. The swelling coating layer is responsible for bursting the outer coating layer (rupturable). This takes place when the swelling layer comes into the contact with water, where an internal force is exerted as a result of the swelling of this layer. The swelling layer may be composed of a disintegrant embedded in a water soluble film forming polymer. A most preferable polymer that can be used as rupturable coating layer is ethylcellulose.

### Therapeutic Uses:

The formulations of the present invention are capable of providing a therapeutically effective amount of a statin, a pharmaceutically acceptable salt or ester thereof or an active form thereof to a subject, for all extended period of time after the start of release. The formulations according to the present invention have increased efficacy and provide at least a similar, if not greater, pharmaceutical effect with the active ingredient, using a significantly decreased dosage amount as compared with conventional formulations known in the art regarding reduce of elevated total cholesterol, low density lipoprotein cholesterol, apolipoprotein B, triglycerides and increase of high density lipoprotein cholesterol. Preferably, the formulations of the present invention contain the statin in an amount that is up to about 60% as compared to an immediate release formulation, yet provides at least similar pharmaceutical efficacy. Thus, the novel formulations of the present invention are more effective than conventional statin formulations currently in use, and can be used for treating high cholesterol, ischemic heart disease and myocardial infarction, or any other disease or condition for which statins are indicated.

The formulations of the present invention may even lead to new indications for the use of delayed burst release of simvastatin and can be used in new populations of patients in which the conventional statin formulations are not used for at present. The formulations of the present invention preferably comprise at least one statin in a decreased dosage amount of up to about 50% as compared to an immediate release formulation of the statin, while providing a substantially equivalent effect of lowering of LDL as a full dosage of the immediate release formulation.

Thus in one aspect, the present invention relates to a method for providing a therapeutically effective amount of a statin, a pharmaceutically acceptable salt or ester thereof or an active form thereof to a subject, comprising orally administering to the subject a controlled release formulation as described herein, featuring a slowly disintegrating core, wherein the formulation releases substantially no statin in vitro for at least about 2 hours to about 6 hours, preferably at least about 2 hours, more preferably at least about 3 hours, also more preferably at least about 4 hours, also more preferably at least about 5 hours and most preferably at least about 6 hours.

According to another embodiment of the present invention, there is provided a delayed onset controlled release formulation for providing an increased bioavailability as measured by AUC of a statin and/or active forms of the statin, relative to that resulting from the administration of an equivalent dose of the conventional immediate release formulations, comprising: a swellable, rapidly disintegrating core comprising at least one statin and at least one release controlling agent and an outer coating over the core, providing delayed release.

According to yet another embodiment of the present invention, such a delayed onset controlled release formulation features an erodible film outer coating over the core, providing delayed release. Optionally the outer coating features a pH dependent film coating. Also optionally and alternatively the outer coating features a combination of a water soluble polymer and/or a water swellable hydrophilic polymer and a water insoluble polymer.

According to other embodiments of the present invention, any of the above described formulations may optionally be used for reducing stress on the liver of the subject treated by at least one other drug involved in liver metabolism when administering a statin.

According to yet other embodiments of the present invention, any of the above described formulations may optionally be used for reducing liver side effects including increased level of transaminases when administering a statin.

According to yet other embodiments of the present invention, any of the above described formulations may optionally be used for reducing muscle pain and/or level of CPK when administering a statin.

According to yet other embodiments of the present invention, any of the above described formulations may optionally be used for reducing gastrointestinal effects comprising one or more of nausea, dyspepsia, flatulence or constipation when administering a statin.

According to yet other embodiments of the present invention, any of the above described formulations may optionally be used for providing release of a statin or a pharmaceutically acceptable salt or ester or active form thereof that is not affected by food intake.

According to still other embodiments of the present invention, any of the above described formulations may optionally be characterized in that the in vivo blood plasma concentration of the statin and/or a pharmaceutically acceptable salt and/or ester thereof is substantially zero for at least about one hour after oral administration and is controlled by the lag time, providing an increased bioavailability as measured by AUC of a statin and/or active forms of said statin, relative to that resulting from the administration of an equivalent dose of the conventional immediate release formulations. Optionally and preferably, the in vivo blood plasma concentration is extended at least 24 hours.

According to still other embodiments of the present invention, any of the above described formulations may optionally be characterized in that the statin is released in the small intestine and/or lower gastrointestinal tract resulting in increased formation of intestinally active forms of the statin.

According to still other embodiments of the present invention, any of the above described formulations may optionally be characterized in that the statin is released in the small intestine and/or lower gastrointestinal tract resulting in an increased concentration of at least one active form in the blood, thereby providing increased bioavailability as measured by AUC. Optionally the formulation comprises a decreased dosage of the statin and/or the pharmaceutically acceptable salt and/or ester thereof. Preferably, the core comprises a dose of statin of no more than about one-half of a dose as compared to a corresponding immediate release formulation, but wherein a level of at least one statin active form after administration of the formulation is at least about a level of the active metabolite after administration of the corresponding immediate release formulation.

### EXAMPLES

The Examples given below are intended only as illustrations of various embodiments of the present invention, and are not intended to be limiting in any way.

### Example 1

### Delayed Onset Controlled Release Formulation

This Example relates to illustrative, non-limiting examples of delayed onset controlled release formulations for statins according to the present invention. For this Example, two different formulations (described as formulations 1A and 1B) were prepared having different cores but coated with the same outer coating, in order to demonstrate the effect of varying different core ingredients on the release profile of the formulation. Both cores are slow release cores, but featuring different amounts of filler ingredients and release controlling agent (in this example, microcrystalline cellulose and HPMC). These variations were shown to affect the release as described in greater detail below. The exact ingredients are given in Table 1 below.

### Preparation of Cores for Formulations 1A and 1B

The cores of Simvastatin 10 mg tablets of samples 1A and 1B were composed from the same granulate ingredients which included: simvastatin, lactose monohydrate (filler), microcrystalline cellulose PH 101 (filler), povidone K 30 (binder). The granulate also featured a plurality of stabilizers, including ascorbic acid, citric acid and BHA. Croscarmelose sodium was featured as disintegrant. The granules were further mixed with other excipients, including, microcrystalline cellulose PH 102 as filler, HPMC K 15 M as release controlling agent, croscarmelose sodium as disintegrant and magnesium stearate as lubricant.

It should be noted that for preparing a granulate, microcrystalline cellulose 101 is preferred as it has a mean particle size of 50 microns. Microcrystalline cellulose (MCC) 102 has a mean particle size of about 100 microns which is mainly useful for direct compression. However, these forms of MCC may optionally both be used for coating (for this exemplary coating, MCC type 102 was used, as it has a lower surface area).

The granulate was prepared by a wet granulation process using a V-Processor. Granulate was milled through 812 micron sieve. Next, the granulate was blended with croscarmelose, HPMC K 15 M and microcrystalline cellulose PH 102 for 30 min. Finally, magnesium stearate which was previously sieved through a sieve with a 600 micron screen, was added into the mixture and blended for additional 2 minutes. The latter process resulted in the tabletting mixture. The tabletting mixture was then compressed with a WICK tabletting press type PR1 equipped with suitable punches diameter 8 mm.

TABLE 1
Core ingredients for formulations 1A and 1B

| | Example 1A | | Example 1B | |
|---|---|---|---|---|
| | | Weight | | Weight |
| | % tablet | (mg/ | % tablet | (mg/ |
| Materials | core | tab) | core | tab) |
| Simvastatin | 3.33% | 10.00 | 3.33% | 10.00 |
| Lactose monohydrate | 6.87% | 20.60 | 6.87% | 20.60 |
| Microcrystalline | 8.33% | 25.00 | 8.33% | 25.00 |
| cellulose Avicel PH 101 | | | | |
| Butylhydroxyanisole | 0.02% | 0.06 | 0.02% | 0.06 |
| (BHA) | | | | |
| Citric acid | 0.67% | 2.00 | 0.67% | 2.00 |
| Ascorbic acid | 1.33% | 4.00 | 1.33% | 4.00 |
| Polyvinylpyrrolidone | 1.00% | 3.00 | 1.00% | 3.00 |
| (Povidone K 30) | | | | |
| Cross-linked carboxy | 0.43% | 1.30 | 0.43% | 1.30 |
| methylcellulose sodium | | | | |
| (Croscarmellose sodium) | | | | |
| Granulation solvent | P. Water, | 0.00 | P. Water, | 0.00 |
| | Isopropanol | | Isopropanol | |
| Hydroxypropyl methyl | 5.00% | 15.00 | 10.00% | 30.00 |
| cellulose (HPMC K | | | | |
| 15 M) | | | | |
| Croscarmellose sodium. | 2.00% | 6.00 | 2.00% | 6.00 |
| Microcrystalline | 70.41% | 211.24 | 65.41% | 196.24 |
| cellulose Avicel PH 102 | | | | |
| Magnesium stearate | 0.60% | 1.80 | 0.60% | 1.80 |
| Total Core | 100.00% | 300.0 | 100.00% | 300.0 |

### Coating for Formulations 1A and 1B

The same coating was used for both formulations and was prepared as follows. Ethyl cellulose was dissolved in ethanol to obtain a clear solution (4.5% w/w), to which cetyl alcohol was added and mixed with the mechanical stirrer to complete dissolution. Microcrystalline cellulose Avicel PH 102 was added and stirred to obtain a homogeneous suspension. The resulting suspension was stirred throughout the whole coating process.

The coating of cores of formulations 1A and 1B was performed in a perforated pan coater using a spraying pressure of 0.4 Bar at 33° C. The coated tablets were dried in oven at 40° C. for about 30 minutes. The coating formulation was as given in Table 2.

TABLE 2
coating for formulations 1A and 1B

| Materials | % of coating | mg/tab |
|---|---|---|
| Microcrystalline cellulose PH 102 | 57.7% | 19.6 |
| Ethyl cellulose 20 | 38.5% | 13.1 |
| Cetyl alcohol | 3.8% | 1.3 |
| Total | 100.0% | 34.0 |

Dissolution tests were performed in apparatus type 1 (baskets), at 37° C., 100 rpm, Medium: 900 ml buffer: 0.1N HCl for 1 hour, then buffer USP pH 7.0 with 0.5% sodium lauryl sulphate (SLS). Samples were automatically drawn from each dissolution cell to test tubes at various time points. Samples were analyzed by a UV (ultraviolet) light detection (238 nm) and analysis device (HPLC). The amount of drug released was calculated according to a standard set of calculations that are known in the art.

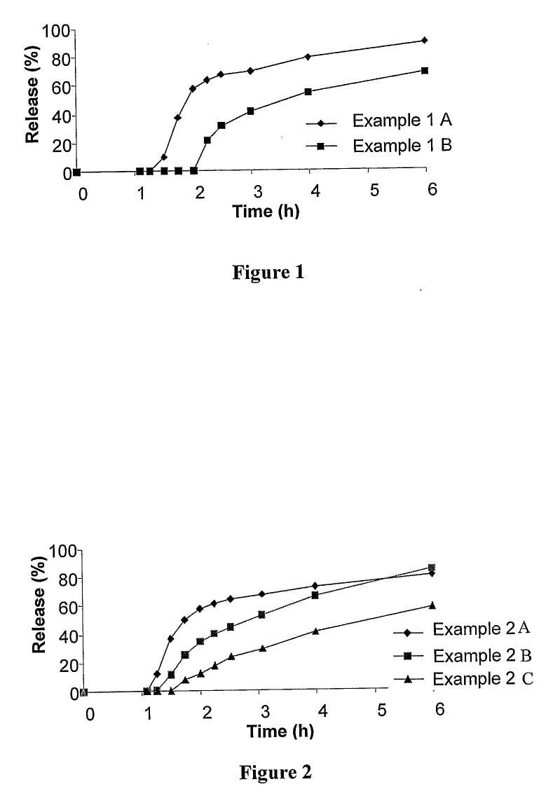

Simvastatin cumulative release (%) from tablets featuring different amounts of binder/filler is given in Table 3 below. The results of simvastatin release (%) from tablets coated with combinations of water insoluble polymer and water insoluble hydrophilic particles are shown graphically in FIG. 1. As shown, increasing the amount of HPMC, with a corresponding decrease in the amount of microcrystalline cellulose Avicel PH 102, caused an increased delay in the onset of release. Without wishing to be limited by a single hypothesis, this may be because increasing the amount of HPMC and decreasing the amount of microcrystalline cellulose in the core formulation may cause formation of a strong hydrogel which eventually leads to a delay in the onset of release.

Overall, this exemplary formulation features a basic delayed disintegrating coating (TCDS), with a combination of hydrophobic film-forming polymer (EC) and water insoluble but hydrophilic and water permeable particles (MCC). Without wishing to be limited by a single hypothesis, coating disintegration may be due to increased osmotic pressure in the core. The core itself features a release controlling agent (which is preferably present in an amount of from about 5 to about 15% w/w relative to the core weight). Delayed release onset is due to the coating.

TABLE 3
Release of Simvastatin from Formulations 1A and 1B

| Hours | Example 1A | Example 1B |
|---|---|---|
| 0.0 | 0.0 | 0.0 |
| 1.08 | 0.0 | 0.0 |
| 1.25 | 0.0 | 0.0 |
| 1.5 | 9.9 | 0.0 |
| 1.75 | 37.1 | 0.0 |
| 2.0 | 57.0 | 0.3 |
| 2.25 | 63.6 | 21.5 |
| 2.5 | 67.1 | 31.2 |
| 3.0 | 69.2 | 41.2 |
| 4.0 | 78.9 | 54.5 |
| 6.0 | 89.2 | 68.0 |

### Example 2

### Delayed Onset Controlled Release Formulation—Additional Examples

This Example relates to additional illustrative, non-limiting examples of delayed onset controlled release formulations for statins according to the present invention. For this Example, three different formulations (described as formulations 2A, 2B and 2C) of 10 mg simvastatin tablets were prepared having different cores but coated with the same outer coating, in order to demonstrate the effect of varying different core ingredients on the release profile of the formulation. All of the cores are slow release cores, but featuring different amounts of release controlling ingredients (in this example, HPMC ranged from about 5% to about 15%). These variations were shown to affect the release as described in greater detail below. For this example, the coating features a combination of a water insoluble polymer and a water soluble polymer.

The exact ingredients are given in Table 4 below.

### Preparation of Cores for Formulations 2A-2C

The same basic granulate was used for formulations 2A-2C of cores of Simvastatin 10 mg tablets according to the present invention. The granulate included: simvastatin, lactose monohydrate (filler), microcrystalline cellulose PH 101 (filler), and povidone K 30 (binder). It also included the following stabilizers: ascorbic acid, citric acid and BHA. It also featured croscarmelose sodium as disintegrant.

The granules were further mixed with other excipients, including: microcrystalline cellulose PH 102 (filler), HPMC K 15 M (release controlling agent), croscarmelose sodium and magnesium stearate as tablet lubricant. The first two ingredients were present in varying amounts as described in greater detail below.

The granulate was prepared by a wet granulation process using a V-Processor. The granulate was milled through 812 micron sieve. Next, the granulate was blended with croscarmelose sodium, HPMC K 15 M and microcrystalline cellulose PH 102 for 30 min. Finally magnesium stearate, which was previously sieved through a sieve with a 600 micron screen, was added into the mixture and blended for additional 2 min. The latter process resulted in a tabletting mixture. The tabletting mixture was then compressed using a WICK tabletting press type PR1 equipped with suitable punches having a diameter of 8 mm.

TABLE 4
Core ingredients for formulations 2A-2C

| | Example 2A | | Example 2B | | Example 2C | |
|---|---|---|---|---|---|---|
| | % tablet | Weight | % tablet | Weight | % tablet | Weight |
| Materials | core | (mg/tab) | core | (mg/tab) | core | (mg/tab) |
| Simvastatin | 3.33% | 10.00 | 3.33% | 10.00 | 3.33% | 10.00 |
| Lactose | 6.87% | 20.60 | 6.87% | 20.60 | 6.87% | 20.60 |
| monohydrate | | | | | | |
| Microcrystalline | 8.33% | 25.00 | 8.33% | 25.00 | 8.33% | 25.00 |
| cellulose PH 101 | | | | | | |
| BHA | 0.02% | 0.06 | 0.02% | 0.06 | 0.02% | 0.06 |
| Citric acid | 0.67% | 2.00 | 0.67% | 2.00 | 0.67% | 2.00 |
| Ascorbic acid | 1.33% | 4.00 | 1.33% | 4.00 | 1.33% | 4.00 |
| Povidone K 30 | 1.00% | 3.00 | 1.00% | 3.00 | 1.00% | 3.00 |
| Croscarmellose | 0.43% | 1.30 | 0.43% | 1.30 | 0.43% | 1.30 |
| sodium | | | | | | |
| Granulation | P. Water, | 0.00 | P. Water, | 0.00 | P. Water, | 0.00 |
| solvent | Isopropanol | | Isopropanol | | Isopropanol | |
| HPMC K 15 M | 5.00% | 15.00 | 10.00% | 30.00 | 15.00% | 45.00 |
| Croscarmellose | 2.00% | 6.00 | 2.00% | 6.00 | 2.00% | 6.00 |
| sodium | | | | | | |
| Microcrystalline | 70.41% | 211.24 | 65.41% | 196.24 | 60.41% | 181.24 |
| cellulose | | | | | | |
| Avicel PH 102 | | | | | | |
| Magnesium | 0.60% | 1.80 | 0.60% | 1.80 | 0.60% | 1.80 |
| stearate | | | | | | |
| Total Core | 100.00% | 300.0 | 100.00% | 300.0 | 100.00% | 300.0 |

### Coating for Formulations 2A-2C

The same coating was used for both formulations and was prepared as follows. Ethyl cellulose was dissolved in ethanol to obtain a clear solution, to which a weighed quantity of kollidon VA 64 (copolyvidone) was added and mixed with the mechanical stirrer to complete dissolution. Sieved talc was added and stirred to obtain a homogeneous suspension. The resulting suspension was stirred throughout the whole coating process.

The coating of cores (formulations 2A, 2B and 2C) was performed in a perforated pan coater with a spraying pressure of 0.4 Bar at 33° C. The coated tablets were dried in oven at 40° C. for about 30 minutes. The coating formulation was as given in Table 5.

TABLE 5
coating for formulations 2A-2C

| Materials | % of coating | mg/tab |
|---|---|---|
| Kollidon VA 64 | 20.0% | 7.4 |
| Ethyl cellulose 20 | 40.0% | 14.8 |
| Talc | 40.0% | 14.8 |
| Total | 100.0% | 37.0 |

Dissolution tests were performed in apparatus type 1 (baskets), at 37° C., 100 rpm, Medium: 900 ml buffer: 0.1N HCl for 1 hour, then buffer USP pH 7.0 with 0.5% SLS. Samples were automatically drawn from each dissolution cell to test tubes at various time points. Samples were analyzed by a UV (ultraviolet) light detection (238 nm) and analysis device (HPLC). The amount of drug released was calculated according to a standard set of calculations that are known in the art.

Simvastatin cumulative release (%) from tablets featuring different amounts of binder/filler is given in Table 6 below. The results of simvastatin release (%) from tablets coated with combinations of water insoluble polymer and water soluble polymer are shown graphically in FIG. 2. As shown, increasing the amount of HPMC, with a corresponding decrease in the amount of microcrystalline cellulose 102, caused an increased delay in the onset of release. Without wishing to be limited by a single hypothesis, this may be because increasing the amount of HPMC and decreasing the amount of microcrystalline cellulose in the core formulation may cause formation of a strong hydrogel which eventually leads to a delay in the onset of release.

Overall, this exemplary formulation features a combination of a water insoluble polymer (EC) and a water soluble polymer (copolyvidone) in the coating. Without wishing to be limited by a single hypothesis, coating disintegration may be due to increased osmotic pressure in the core. The core itself features a release controlling agent (which is preferably present in an amount of from about 5 to about 15% w/w relative to the core weight). Delayed release onset is due to the coating.

TABLE 6
Release of Simvastatin from Formulations 2A-2C

| Hours | Example 2A | Example 2B | Example 2C |
|---|---|---|---|
| 0.0 | 0.0 | 0.0 | 0.0 |
| 1.1 | 0.0 | 0.0 | 0.0 |
| 1.3 | 12.0 | 0.6 | 0.0 |
| 1.5 | 36.4 | 11.7 | 0.0 |
| 1.8 | 50.0 | 25.4 | 8.0 |
| 2.0 | 57.4 | 34.4 | 12.0 |
| 2.3 | 61.2 | 39.9 | 17.6 |
| 2.5 | 63.8 | 44.4 | 24.0 |
| 3.0 | 67.0 | 52.8 | 29.0 |
| 4.0 | 72.7 | 65.7 | 40.5 |
| 6.0 | 79.9 | 83.7 | 57.3 |

### Example 3

### Delayed Onset Controlled Release Formulation with Enteric Coating

This Example relates to an illustrative, non-limiting example of a delayed onset controlled release formulations for statins according to the present invention, featuring a slow release core coated with an enteric coating.

The exact ingredients are given in Table 7 below.

### Preparation of Cores for Formulation 3A

The core of Simvastatin 10 mg tablets for formulation 3A was composed from the same basic granulate as for both previous Examples. The granules were prepared by wet granulation process using a V-Processor.

Next, the granulate was blended with HPMC K 15 M and microcrystalline cellulose PH 102 for 30 min. Finally magnesium stearate which was previously sieved through a sieve with a 600 micron screen was added into the mixture and blended for additional 2 minutes. The latter process resulted in a tabletting mixture. The tabletting mixture was then compressed using a WICK tabletting press type PR1 equipped with suitable punches diameter 7 mm.

TABLE 7
core ingredients for formulation 3A

| Materials | % tablet core | Weight (mg/tab) |
|---|---|---|
| Simvastatin | 5.00% | 10.00 |
| Lactose monohydrate | 10.30% | 20.60 |
| Microcrystalline cellulose PH 101 | 12.50% | 25.00 |
| BHA | 0.03% | 0.06 |
| Citric acid | 1.00% | 2.00 |
| Ascorbic acid | 2.00% | 4.00 |
| Povidone K 30 | 1.50% | 3.00 |
| Croscarmellose sodium | 0.65% | 1.30 |
| Granulation solvent | P. Water, | 0.00 |
| | Isopropanol | |
| HPMC K 15 M | 10.00% | 20.00 |
| Microcrystalline cellulose PH 102 | 56.42% | 112.84 |
| Magnesium stearate | 0.60% | 1.20 |
| Total Core | 100.00% | 200.0 |

### Coating for Formulation 3A

Triethyl Citrate was dissolved in water to obtain a clear solution, then sodium lauryl sulphate was added to the resulting solution with slow stirring. Hydroxypropylmethyl cellulose acetate succinate (HPMC AS) was added to obtain an aqueous dispersion. Sieved Talc was added and stirred to obtain a homogeneous suspension. The resulting suspension was stirred throughout the whole coating process.

The coating of cores was performed in a perforated pan coater with a spraying pressure of 1.5-2 Bar at 40° C. The coated tablets were dried in oven at 50° C. for about 2 hours. The coating formulation was as given in Table 8 below.

TABLE 8
coating for formulation 3A

| Coating | % of coating | mg/tab |
|---|---|---|
| HPMC AS | 55.2% | 40.8 |
| Triethyl citrate | 15.5% | 11.5 |
| Sodium lauryl sulphate | 1.7% | 1.3 |
| Talc | 27.6% | 20.4 |
| Total | 100.0% | 74.0 |

Dissolution tests were performed in apparatus type 1 (baskets), at 37° C., 100 rpm, Medium: 900 ml buffer: 0.1N HCl for 1 hour, then buffer USP pH 7.0 with 0.5% SLS. Samples were automatically drawn from each dissolution cell to test tubes at various time points. Samples were analyzed by a UV (ultraviolet) light detection (238 nm) and analysis device (HPLC). The amount of drug released was calculated according to a standard set of calculations that are known in the art.

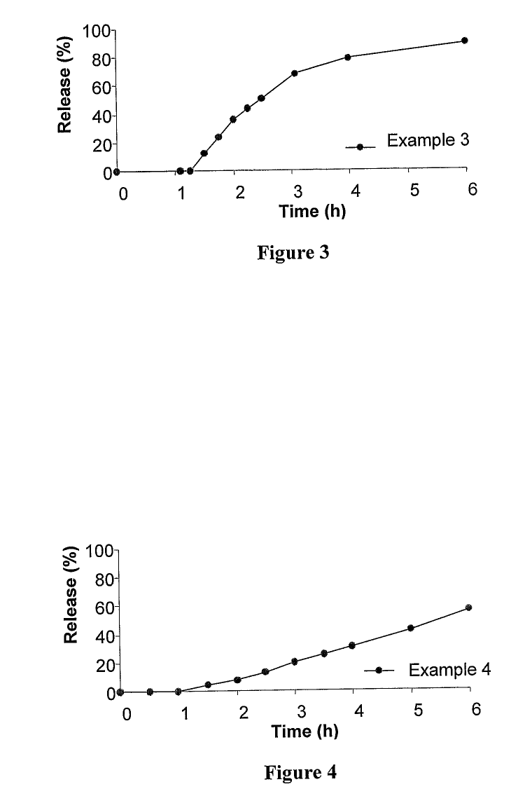

Simvastatin cumulative release (%) from tablets coated with an enteric coating is given in Table 9 below. The results of simvastatin release (%) from enteric coated tablets (10% HPMC in cores) are shown graphically in FIG. 3. As shown, coating the tablets with an enteric coating resulted in a steeper rate of release of simvastatin, as this formulation demonstrates pH controlled release.

Overall, this exemplary formulation features an enteric coating which has pH controlled release, as coating disintegration is pH dependent. The core itself features a release controlling agent (which is preferably present in an amount of from about 5 to about 15% w/v relative to the core weight) but specifically does not feature a disintegrant.

TABLE 9
Release of Simvastatin from Formulation 3A

| Hours | Example 3 |
|---|---|
| 0.0 | 0.0 |
| 1.1 | 0.0 |
| 1.3 | 0.0 |
| 1.5 | 12.3 |
| 1.8 | 24.0 |
| 2.0 | 36.2 |
| 2.3 | 43.5 |
| 2.5 | 50.4 |
| 3.1 | 68.0 |
| 4.0 | 78.6 |
| 6.0 | 89.3 |

### Example 4

### Delayed Onset Controlled Release Formulation with Dry Coating

This Example relates to an illustrative, non-limiting example of a delayed onset controlled release formulations for statins according to the present invention, featuring a slow release core coated with dry coating.

The exact ingredients are given in Table 10 below.

### Preparation of Cores for Formulation 4A

The core of Simvastatin 10 mg tablets for formulation 4A was composed from the same basic granulate as for the previous Examples. The granules were prepared by wet granulation process using a V-Processor.

Next, the granulate was blended with HPMC K 15 M and microcrystalline cellulose PH 102 for 30 min. Finally magnesium stearate which was previously sieved through a sieve with a 600 micron screen was added into the mixture and blended for additional 2 minutes. The latter process resulted in a tabletting mixture. The tabletting mixture was then compressed using a WICK tabletting press type PR1 equipped with suitable punches diameter 7 mm.

TABLE 10
core ingredients for formulation 4A

| Materials | % tablet core | Weight (mg/tab) |
|---|---|---|
| Simvastatin | 5.00% | 10.00 |
| Lactose monohydrate | 10.30% | 20.60 |
| Microcrystalline cellulose | 12.50% | 25.00 |
| Avicel PH 101 | | |
| BHA | 0.03% | 0.06 |
| Citric acid | 1.00% | 2.00 |
| Ascorbic acid | 2.00% | 4.00 |
| Povidone K 30 | 1.50% | 3.00 |
| Croscarmellose sodium | 0.65% | 1.30 |
| Granulation solvent | P. Water, | 0.00 |
| | Isopropanol | |
| HPMC K 15 M | 10.00% | 20.00 |
| Microcrystalline cellulose | 56.42% | 112.84 |
| Avicel PH 102 | | |
| Magnesium stearate | 0.60% | 1.20 |
| Total core | 100.00% | 200.0 |

### Coating for Formulation 4A

Lactose was mixed with HPMC K 15 M for 30 min. Sieved magnesium stearate (using a sieve with a 600 micron screen) was added into the mixture and blended for 2 min. The latter process resulted in the tabletting mixture which was used for the dry coating of the inner core (active material containing core, diameter 7 mm). The dry coating was performed on the inner core using a WICK tabletting press type PR1 equipped with suitable punches with diameter 10 mm, so that the inner tablet was placed in the middle of a outer tablet (dry coat) with the total average weight 550 mg and average hardness (of the outer tablet) 790 N.

The coating formulation was as given in Table 11 below.

TABLE 11
coating for formulation 4A

| Materials | % tablet core | Weight (mg/tab) |
|---|---|---|
| HPMC K 15 M | 10.0% | 35.0 |
| Lactose monohydrate | 89.4% | 312.9 |
| Magnesium stearate | 0.60% | 2.1 |
| Total dry coating | 100.00% | 350.0 |

Dissolution tests were performed using an apparatus type 1 (baskets), at 37° C., 100 rpm. Medium: 900 ml, buffer USP pH 7.0 with 0.5% SLS. Samples were automatically drawn from each dissolution cell to test tubes at various time points. Samples were analyzed by a UV (ultraviolet) light detection (238 nm) and analysis device (HPLC). The amount of drug released was calculated according to a standard set of calculations that are known in the art.

Simvastatin cumulative release (%) from tablets coated with a dry coating is given in Table 12 below. The results of simvastatin release (%) from dry coated tablets (10% HPMC in cores and coating) are shown graphically in FIG. 4. As shown, coating the tablets with a dry coating resulted in a slower, more gradual rate of release of simvastatin, as release onset for this formulation depends on the degradation rate of the outer coating.

Overall, this exemplary formulation features a dry coating with a hydrogel forming polymer that preferably has a slow degradation rate. The core features a release controlling agent (which is preferably present in an amount of from about 5 to about 15% w/w relative to the core weight) but specifically does not feature a disintegrant.

TABLE 12
Release of Simvastatin from Formulation 4A

| Hours | Example 4 |
|---|---|
| 0.0 | 0.0 |
| 0.5 | 0.0 |
| 1.0 | 0.0 |
| 1.5 | 4.6 |
| 2.0 | 8.0 |
| 2.5 | 13.0 |
| 3.0 | 20.0 |
| 3.5 | 25.2 |
| 4.0 | 30.5 |
| 5.0 | 42.0 |
| 6.0 | 56.0 |

### Example 5

### Delayed Onset Controlled Release Formulation with Coating Featuring Water Insoluble Flexible Polymer and Water Insoluble Hydrophilic Particles

This Example relates to an illustrative, non-limiting example of a delayed onset controlled release formulations for statins according to the present invention, featuring a swellable core coated with a coating comprising water insoluble flexible polymer and water insoluble hydrophilic particles.

The exact ingredients are given in Table 13 below.

### Preparation of Cores for Formulation 5A

The core of Simvastatin 10 mg tablets for formulation 5A was composed from the same basic granulate as for the previous Examples. The granules where further mixed with other excipients, including: microcrystalline cellulose PH 102 (filler), silicon dioxide (wicking controlling agent, which enables the core to be swellable in combination with a swellable polymer), croscarmelose sodium (disintegrant) and magnesium stearate (lubricant).

The granulate was prepared by a wet granulation process using a V-Processor. The granulate was milled through 812 micron sieve. Next, the granulate was blended with croscarmelose, and microcrystalline cellulose for 5 min. The resulting mixture was blended with Silicon dioxide and the rest amount of microcrystalline cellulose for 30 minutes. Finally, magnesium stearate which was previously sieved through a sieve with a 600 micron screen, was added into the mixture and blended for additional 2 minutes. The latter process resulted in the tabletting mixture. The tabletting mixture was then compressed with a KILIAN tabletting press equipped with suitable punches, to provide caplet-shaped tablets having a first dimension of 6 mm and a second dimension of 12 mm.

TABLE 13
core ingredients for formulation 5A

| | Sample 5 | |
|---|---|---|
| | | Weight |
| Materials | % tablet core | (mg/tab) |
| Simvastatin | 3.33% | 10.00 |
| Lactose monohydrate | 6.87% | 20.60 |
| Microcrystalline cellulose Avicel PH 101 | 8.33% | 25.00 |
| BHA | 0.02% | 0.06 |
| Citric acid | 0.67% | 2.00 |
| Ascorbic acid | 1.33% | 4.00 |
| Povidone K 30 | 1.00% | 3.00 |
| Croscarmellose sodium | 0.43% | 1.30 |
| Granulation solvent | P. Water, | 0.00 |
| | Isopropanol | |
| Colloidal silicon dioxide | 2.00% | 6.00 |
| Croscarmellose sodium | 2.00% | 6.00 |
| Microcrystalline cellulose Avicel PH 102 | 73.41% | 220.24 |
| Magnesium stearate | 0.60% | 1.80 |
| Total core | 100.00% | 300.0 |

### Preparation of Coating for Formulation 5A

Eudragit E 100 was dissolved in ethanol to obtain a clear solution (10% w/w). Microcrystalline cellulose Avicel PH 102 was added and stirred to obtain a homogeneous suspension, which was kept stirred during the whole coating process.

The coating of the cores was performed in a perforated pan coater using a spraying pressure of 0.4 Bar at 33° C. The coated tablets were dried in oven at 40° C. for about 30 minutes. The coating formulation is given in Table 14 below. It should be noted that Eudragit E 100 is the water insoluble flexible polymer in this example, while microcrystalline cellulose Avicel PH 102 forms the water insoluble hydrophilic particles.

TABLE 14
coating for formulation 5A

| Materials | % of coating | mg/tab |
|---|---|---|
| Microcrystalline cellulose | 50.0% | 18.0 |
| Avicel PH 102 | | |
| Eudragit E 100 | 50.0% | 18.0 |
| Total | 100.0% | 36.0 |

Dissolution tests were performed using an apparatus type 1 (baskets), at 37° C., 100 rpm. Medium: 900 ml, buffer USP pH 7.0 with 0.5% SLS. The sample was stirred with a VanKel basket stirrer (Van Kel Inc., USA). Samples were automatically drawn from each dissolution cell to test tubes at various time points. Samples were analyzed by a UV (ultraviolet) light detection (238 nm) and analysis device (HPLC). The amount of drug released was calculated according to a standard set of calculations that are known in the art.

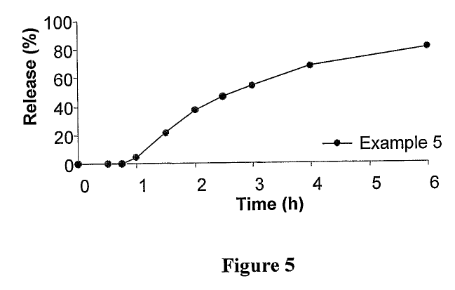

Simvastatin cumulative release (%) from tablets coated with a coating featuring a water insoluble polymer and particles of a hydrophilic but water insoluble polymer is given in Table 15 below. The results of simvastatin release (%) from tablets coated with combination of water insoluble flexible polymer and water insoluble hydrophilic particles are shown graphically in FIG. 5. As shown, coating the tablets with a coating featuring the water insoluble polymer and the particles resulted in a more gradual rate of release of simvastatin with an earlier onset, as this formulation demonstrates diffusion-controlled (time-controlled) release.

Overall, this exemplary formulation features a combination of a water insoluble flexible polymer (Eudragit E) and water insoluble hydrophilic water permeable particles (MCC). The formulation has diffusion controlled release. The core is a swellable core, featuring a swellable polymer and a wicking controlling agent, which controls entry of liquid to the swellable polymer.

TABLE 15
Release of Simvastatin from Formulation 5A

| Hours | Example 5 |
|---|---|
| 0.0 | 0.0 |
| 0.5 | 0.0 |
| 0.75 | 0.0 |
| 1.0 | 4.6 |
| 1.5 | 21.4 |
| 2.0 | 37.4 |
| 2.5 | 46.6 |
| 3.0 | 54.2 |
| 4.0 | 68.0 |
| 6.0 | 80.9 |

### Bioavailability Study

A randomized, pharmacokinetic pilot study is undertaken to evaluate the bioavailability of test formulations of HMG-CoA reductase inhibitors (statins), and also to determine the levels of the main metabolite, if relevant. For example the study is performed on simvastatin and its main metabolite simvastatin hydroxy acid. For the study, 10 mg simvastatin tablets are prepared according to any suitable example above.

### Efficacy Study

The formulation of the present invention is believed to have increased efficacy and to be capable of providing at least similar, if not greater, pharmaceutical effects with the active ingredient with a significantly decreased dosage amount as compared to other orally administered formulations that are known in the art. Without wishing to be limited by a single hypothesis, it is also possible that lower side effects may be observed with the formulation of the present invention, again as compared to other orally administered dosage forms that are known in the art.

Optionally, such a decreased dosage amount of the active ingredient, preferably simvastatin, comprises up to about 60% of the regular dosage amount, more preferably up to about 50% and most preferably up to about 40% of the regular dosage amount. Optionally, the dosage amount comprises up to about 30% of the regular dosage amount. One non-limiting example of a “regular” dosage amount is that administered with the currently available reference product, which as noted above is the Zocor® (immediate release) product of Merck. Any other immediate release product could also be considered to be a “regular” product that is known in the art. The dosage amount during a 24 hour period is also determined by the dosage frequency; preferably, the formulation of the present invention is not administered more frequently than the “regular” orally administered formulations; more preferably, the formulation of the present invention is administered once daily, optionally in the evening.

A clinical study studies the issue of both pharmaceutical efficacy as well as bioavailability. This study compares the efficacy and pharmacokinetic parameters of a tablet according to the present invention, with the Zocor® reference product (also as used in the bioavailability studies above) which contains a regular dosage of simvastatin. The clinical study is conducted with patients suffering from hypercholesterolemia, although it should be noted that this is for the purpose of the study only and is not intended to be limiting in any way.

The primary end point criteria of the study is equivalent or superior mean percent reductions from baseline (i.e. before the study) in LDL-C (LDL (low density lipoprotein) concentrations in the blood) observed in patients taking the tablet according to the present invention, as compared to the reference product.

Both sets of patients take one tablet per day (present invention or reference) in the evening. Each set includes 80 patients having elevated cholesterol levels. The patients either have not been previously treated with a statin, or are undergoing a 6 week washout period (during which no statin is given) before the study begins. The study is double-blind, randomized and multicenter. Any potential adverse effects are detected with clinical and laboratory testing.

A treatment period of 6 weeks occurs, with periodic measurements of the blood.

As noted above, the clinical study shows that the tablet of the present invention (with the lower dosage amount of 10 to 12 mg per tablet) is at least as pharmaceutically effective as the immediate release reference product (with 20 mg per tablet), thereby providing at least similar clinical efficacy but with a significantly lower dose (up to about 40% of the immediate release reference product).

Treatment with the tablet of the present invention has at least similar effects as in published literature/studies, although the final comparison is made with the set of patients who are taking the reference product, Zocor, within the study itself.

The formulation of the present invention therefore provides a delayed onset, controlled release formulation for delivery of statins in the lower GI tract preferentially to the colon or small intestine, which provides higher blood levels of statin or its metabolites in the bloodstream in comparison to a conventional immediate release formulation. The bioavailability is shown to be higher than that of a known reference product. The formulations according to the present invention should result in fewer side effects, greater safety, efficacy, and patient compliance.

The formulation of the present invention preferably comprises a delayed onset, controlled release formulation, which is not a delayed burst release or delayed immediate or fast release formulation. The release is designed to occur within a period of less than 8 hours following oral administration, preferably with selective absorption of the active agent in the lower GI tract.

It is appreciated that certain features of the invention, which are, for clarity, described in the context of separate embodiments, may also be provided in combination in a single embodiment, Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the air. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1.-180. (canceled)

181. A delayed onset slow release formulation for a statin and/or active forms of the statin, comprising: a slowly disintegrating core comprising at least one statin, a filler and a release controlling agent, wherein the release controlling agent is present in a range of from about 5% to about 20% weight per weight relative to the total weight of the core, and a rate-controlling outer coating over the core, the rate-controlling outer coating selected from the group consisting of:
i) an outer coating featuring a mixture of cellulosic water insoluble polymers;
ii) an outer coating featuring a combination of a water insoluble polymer and a water soluble polymer;
iii) an outer coating featuring an enteric polymer coating;
iv) an outer coating featuring a polymer disintegrating at pH values above about pH 5;
v) an outer coating featuring a cellulosic polymer that is applied as a dry coating, the polymer forming a hydrogel;
vi) an outer coating featuring water insoluble hydrophilic particles embedded in a water insoluble flexible polymer, wherein the statin and/or active forms of the statin are released through diffusion through the water insoluble hydrophilic particles; and
vii) an outer coating featuring a dry coating or an enteric coating, with the proviso that the core does not comprise a disintegrating agent.

182. The formulation of claim 181, wherein the mixture of cellulosic water insoluble polymers comprises any two or more polymers selected from the group consisting of HPMC, EC, and microcrystalline cellulose.

183. The formulation of claim 181, wherein the combination of the water insoluble polymer and the water soluble polymer comprises a water insoluble polymer selected from the group consisting of a podimethylaminoethylacrylate/ethylmethacrylate copolymer, the copolymer being based on acrylic and methacrylic acid esters with a low content of quaternary ammonium groups, wherein the molar ratio of the ammonium groups to the remaining neutral (meth)acrylic acid esters is approximately 1:20, the polymer corresponding to USP/NF Ammonio Methacrylate Copolymer Type A, an ethylmethacrylate/chlorotrimethylammoniumethyl methacrylate copolymer, the copolymer based on acrylic and methacrylic acid esters with a low content of quaternary ammonium groups wherein the molar ratio of the ammonium groups to the remaining neutral (meth)acrylic acid esters is 1:40, the polymer corresponding to USP/NF Ammonio Methacrylate Copolymer Type B, a dimethylaminoethylmethacrylate/methylmethacrylate and butylmethacrylate copolymer, a copolymer based on neutral methacrylic acid esters and dimethylaminoethyl methacrylate esters wherein the polymer is cationic in the presence of acids, an ethylacrylate and methylacrylate/ethylmethacrylate and methyl methylacrylate copolymer, the copolymer being a neutral copolymer based on neutral methacrylic acid and acrylic acid esters, ethylcellulose, shellac, zein, and waxes, paraffin, cellulose acetate, cellulose propionate, cellulose acetate propionate, cellulose acetate butyrate, cellulose acetate phthalate, cellulose triacetate, poly (methyl methacrylate), poly(ethylmethacrylate), poly (butyl methacrylate), poly (isobutyl methacrylate), and poly (hexyl methacrylate), poly (isodecyl methacrylate), poly(lauryl methacrylate), poly (phenyl methacrylate), poly (methylacrylate), poly (isopropyl acrylate), poly (isobutyl acrylate) poly(octadecyl acrylate), poly (ethylene), poly (ethylene) low density, poly(ethylene) high density, poly (ethylene oxide), poly (ethyleneterephthalate), poly (vinyl isobutyl ether), poly (vinyl acetate), poly(vinyl chloride) and polyurethane, or mixtures thereof.

184. The formulation of claim 183, wherein the water insoluble polymer comprises ethylcellulose.

185. The formulation of claim 181, wherein the water soluble polymer comprises polyvinyl alcohol, polyvinylpyrrolidone (PVP), copolyvidone, methylcellulose, hydroxypropylcellulose, hydroxypropylmethyl cellulose, polyethylene glycol, carboxymethyl cellulose (sodium salt), hydroxyethyl cellulose, a water soluble gum, polysaccharide and/or mixtures thereof.

186. The formulation of claim 185, wherein the water soluble polymer comprises copolyvidone.

187. The formulation of claim 181, wherein the enteric polymer coating comprises a polymer selected from the group consisting of cellulose acetate phthalate, hydroxy propyl methyl cellulose acetate succinate, poly(methacrylic acid, methyl methacrylate)1:1 and (Eudragit L100), poly(methacrylic acid, ethyl acrylate)1:1 (Eudragit L30D-55).

188. The formulation of claim 187, wherein the polymer comprises HPMC AS.

189. The formulation of claim 181, wherein the cellulosic polymer of the dry coating comprises HPMC K 15 M, or wherein the water insoluble flexible polymer comprises a methacrylic polymer.

190. The formulation of claim 189, wherein the methacrylic polymer comprises Eudragit E.

191. The formulation of claim 189, wherein the water insoluble hydrophilic particles comprise a polymer forming a hydrogel upon contact with liquid, the polymer being selected from the group consisting of poly(hydrox alkyl methacrylate); kappa-carrageenan; polyvinylpyrrolidone; anionic and cationic hydrogels; polyelectrolyte complexes; poly(vinyl alcohol) having low amounts of acetate, cross-linked with glyoxal, formaldehyde, or glutaraldehyde; a mixture comprising methyl cellulose, cross-linked agar and carboxymethyl cellulose; a water-insoluble, water-swellable copolymer produced by forming a dispersion of finely divided maleic anhydride with styrene, ethylene, propylene, butylene or isobutylene; water-swellable polymers of N-vinyl lactams; polysaccharide, water swellable gums, high viscosity hydroxylpropylmethyl cellulose or mixtures thereof.

192. The formulation of claim 191, wherein the water insoluble polymer is calcium pectinate or microcrystalline cellulose.

193. The formulation of claim 181, wherein the filler is present in an amount ranging from about 10% to about 85% (W/W) relative to the total weight of core, and is selected from the group consisting of starch, lactitol, lactose, an inorganic calcium salt, microcrystalline cellulose, sucrose, and combinations thereof.

194. The formulation of claim 193, wherein the microcrystalline cellulose has a particle size of less than about 100 microns.

195. The formulation of claim 181, wherein the release controlling agent comprises a water insoluble, hydrophilic swellable polymer selected from the group consisting of poly(hydrox alkyl methacrylate); kappa-carrageenan; polyvinylpyrrolidone; anionic and cationic hydrogels; polyelectrolyte complexes; poly(vinyl alcohol) having low amounts of acetate, cross-linked with glyoxal, formaldehyde, or glutaraldehyde; a mixture comprising methyl cellulose, cross-linked agar and carboxymethyl cellulose; a water-insoluble, water-swellable copolymer produced by forming a dispersion of finely divided maleic anhydride with styrene, ethylene, propylene, butylene or isobutylene; water-swellable polymers of N-vinyl lactams; polysaccharide, water swellable gums, high viscosity hydroxylpropylmethyl cellulose and mixtures thereof.

196. A delayed onset controlled release formulation for providing an increased bioavailability as measured by AUC of a statin and/or active forms of the statin, relative to that resulting from the administration of an equivalent dose of the conventional immediate release formulations, comprising: a slowly disintegrating core comprising at least one statin and at least one release controlling agent and a rate-controlling outer coating over the core, providing controlled release.

197. The formulation of claim 196, wherein the release controlling agent comprises a material selected from the group consisting of a suitable hydrophilic cellulosic gelling polymer; vinyl polymers; acrylic polymers and copolymers, natural and synthetic gums, gelatin, collagen, proteins, polysaccharides; and mixtures thereof.

198. The formulation of claim 197, wherein the polysaccharide in the release controlling agent comprises a cross-linked polysaccharide selected from the group consisting of insoluble metal salts or cross-linked derivatives of alginate, pectin, xantham gum, guar gum, tragacanth gum, and locust bean gum, carrageenan, metal salts thereof, and covalently cross-linked derivatives thereof, or wherein the cellulosic polymer comprises a modified cellulose selected from the group consisting of cross-linked derivatives of hydroxypropylcellulose, hydroxypropylmethylcellulose, hydroxyethylcellulose, methylcellulose, carboxymethylcellulose, and metal salts of carboxymethylcellulose.

199. The formulation of claim 197, wherein the cellulosic gelling polymer comprises a material selected from the group consisting of methylcellulose, carboxymethylcellulose, hydroxypropylcellulose, hydroxypropylmethylcellulose, hydroxyethylcellulose; or wherein the vinyl polymer is selected from the group consisting of polyvinylpyrrolidone, polyvinyl alcohol, and the like.

200. The formulation of claim 197 wherein the acrylic polymers and copolymers are selected from the group consisting of acrylic acid polymer, methacrylic acid copolymers, ethyl acrylate-methyl methacrylate copolymers; or wherein the natural and synthetic gums are selected from the group consisting of guar gum, arabic gum, xanthan gum; or wherein the polysaccharides are selected from the group consisting of pectin, pectic acid, alginic acid, sodium alginate, polyaminoacids, polyalcohols and polyglycols.

201. The formulation of claim 187, selected from:
a) a formulation that preferentially releases statin in the intestine of the subject;
b) a formulation that preferentially release statin in the lower gastrointestinal tract; and
c) a formulation that preferentially releases statin in the colon of the subject.

202. The formulation of claims 196, wherein the core is in the form of one of a tablet, pellets, microparticles, agglomerate, and capsule.

203. The formulation of claim 196, wherein the release controlling agent is present in an amount ranging from about 5% to about 20% weight per weight of the core.

204. The formulation of claim 196, wherein the core further comprises at least one of a filler, an absorption enhancer, a binder, a disintegrant, a hardness enhancing agent, a stabilizer, a flow regulating agent, a lubricant, an antioxidant, a chelating agent, a sequestrate and a wicking agent.

205. The formulation of claim 196, wherein the coating comprises at least one of the following:
a. pH dependent polymer
b. a combination of at least one water soluble polymer and at least one water insoluble polymer
c. a combination of at least one swellable polymer and at least one water insoluble polymer
d. a combination of at least a water soluble pore forming agent and at least one water insoluble polymer
e. at least one swellable gel forming polymer
g. an erodible composition
h. a combination of at least one pH dependent polymer and at least one water insoluble polymer

206. A delayed onset controlled release formulation according to claim 16, selected from the group consisting of:
i) a formulation comprising: a swellable, slowly disintegrating core comprising at least one statin, at least one, hydrophilic, swellable, hydrogel-forming material and a wicking controlling agent, and a rate controlling outer coating over the core comprising a water permeable agent through which fluid enters the core;
ii) a formulation comprising: a swellable, slowly disintegrating core comprising at least one statin and at least one release controlling agent and an outer coating over the core, providing delayed release, characterized in that the in vivo blood plasma concentration of the statin and/or a pharmaceutically acceptable salt and/or ester thereof in the subject is substantially zero for at least about two hours after oral administration;
iii) a formulation comprising: a core comprising at least one statin and at least one release controlling agent and an erodible film outer coating over the core, providing delayed release;
iv) a formulation comprising: a slowly disintegrating core comprising at least one statin and at least one release controlling agent, and a pH dependent film outer coating over the core, providing delayed release; and
v) a formulation comprising: a slowly disintegrating core comprising at least one statin and at least one release controlling agent and an outer coating over the core, comprising a combination of a water soluble polymer and/or a water swellable hydrophilic polymer and a water insoluble polymer.

207. The formulation of claim 196, wherein the formulation releases substantially no statin in vitro for at least about 2 to about 6 hours.

208. The formulation of claim 196, wherein the statin is selected from the group comprising simvastatin, beta-hydroxy acid simvastatin, lovastatin, mevastatin, pravastatin, fluvastatin, atorvastatin, pitavastatin, rivastatin and cerivastatin, or pharmaceutically acceptable salts and esters thereof.

209. A method for using a formulation according to claim 181 to reduce stress on the liver of the subject treated by at least one other drug involved in the liver metabolism, to reduce stress on the liver of the subject, to reduce liver side effects including increased level of transaminases, to reduce muscle pain and level of CPK, to reduce gastrointestinal effects comprising nausea, dyspepsia, flatulence and constipation, or for providing treatment for high blood cholesterol to a subject in need thereof.

210. A method for using a formulation according to claim 196, to reduce stress on the liver of the subject treated by at least one other drug involved in the liver metabolism, to reduce stress on the liver of the subject, to reduce liver side effects including increased level of transaminases, to reduce muscle pain and level of CPK, to reduce gastrointestinal effects comprising nausea, dyspepsia, flatulence and constipation, or for providing treatment for high blood cholesterol to a subject in need thereof.

211. A method for using a formulation according to claim 181, for providing release of the statin or the pharmaceutically acceptable salt or ester or active form thereof that is not affected by food.

212. A method for using a formulation according to claim 196, for providing release of the statin or the pharmaceutically acceptable salt or ester or active form thereof that is not affected by food.

213. A method for providing a therapeutically effective amount of a statin or a pharmaceutically acceptable salt or ester or active form thereof to a subject, comprising administering orally to the subject a formulation according to claim 181.

214. A method for providing a therapeutically effective amount of a statin or a pharmaceutically acceptable salt or ester or active form thereof to a subject, comprising administering orally to the subject a formulation according to claim 196.

215. A method for providing enhanced bioavailability of a statin or pharmaceutically acceptable salts or esters thereof or its related metabolite in a subject, or for providing an increased amount of a statin or a pharmaceutically acceptable salt or ester thereof, relative to that resulting from the administration of an equivalent dose of the conventional immediate release formulations, comprising:
administering orally to the subject a delayed onset controlled release formulation comprising a core and an outer coating that surrounds the core;
the core comprising a statin, or a pharmaceutically acceptable salt thereof and at least one release controlling agent, and
the coating characterized by at least one of
a. pH dependent coating film, preferably an enteric coating
b. a combination of at least one water soluble polymer and at least one water insoluble polymer
c. a combination of at least one swellable polymer and at least one water insoluble polymer
d. a combination of at least a water soluble pore forming agent and at least one water insoluble polymer
e. at least one swellable gel forming polymer
f. at least one erodible polymer
g. a combination of at least one pH dependent polymer and at least one water insoluble polymer
h. a two-layer coating comprising a rupturable outer layer and swellable inner layer wherein the in vivo blood plasma concentration of the statin is substantially zero for at least about two hours after oral administration.

216. A delayed onset controlled release formulation according to claim 196 that releases a statin and/or a pharmaceutically acceptable salt and/or ester thereof in the lower gastrointestinal tract of a subject, characterized in that the in vivo blood plasma concentration of the statin and/or a pharmaceutically acceptable salt and/or ester thereof is substantially zero for at least about one hour after oral administration and is controlled by the lag time, providing an increased bioavailability of a statin and/or active forms of the statin, as measured by AUC relative to that resulting from the administration of an equivalent dose of the conventional immediate release formulations.

217. The formulation of claim 216,
wherein the in vivo blood plasma concentration is extended at least 24 hours, or
wherein the core comprises a dose of statin of no more than about one-half of a dose as compared to a corresponding immediate release formulation, but wherein a level of at least one statin active form after administration of the delayed controlled release formulation is at least about a level of the active metabolite after administration of the corresponding immediate release formulation, or
wherein the core comprises a dose of statin of no more than about one-half of a dose in the corresponding immediate release formulation, but wherein a level of at least one statin active metabolite after administration of the controlled release formulation is at least about a level of the active metabolite after administration of the corresponding immediate release formulation.

218. A method for providing enhanced bioavailability of statin and/or a pharmaceutically acceptable salts and/or esters thereof and/or its related metabolite in a subject, comprising: administering orally to the subject a controlled release formulation excluding delayed burst release and delayed immediate or fast release characterized in that the in vivo blood plasma concentration of the statin is substantially zero for at least about one hour after oral administration.

219. A controlled release method for providing an increased amount of a statin and/or active forms of the statin circulating in the blood of a subject, relative to that resulting from the administration of an equivalent dose of a conventional immediate release formulation to the subject, wherein the method excludes delayed burst release and delayed immediate or fast release.
